(19) United States
(12) Patent Application Publication
(10) Pub. No.: US 2010/0129800 A1
(43) Pub. Date: May 27, 2010

(54) POLYMERS AND THEIR USE AS FLUORESCENT LABELS

(76) Inventors: Juan Aymami Bofarull, Barcelona (ES); Fernando Albericio Palomera, Barcelona (ES); Anna Maria Avino Andres, Barcelona (ES); Josep Farrera Sinfreu, Lleida (ES); Miriam Royo Exposito, Barcelona (ES); Isabel Navarro Munoz, Barcelona (ES); Ramon Eritja Casadella, Barcelona (ES)

Correspondence Address: TRASKBRITT, P.C., P.O. BOX 2550, SALT LAKE CITY, UT, 84110, US

(21) Appl. No.: 12/452,626

(22) PCT Filed: Jul. 9, 2008
(86) PCT No.: PCT/EP2008/058922

§ 371 Date: Jan. 11, 2010

(30) Foreign Application Priority Data
Jul. 12, 2007 (EP) 07112386.3

(51) Int. Cl. C12Q 1/68; C07D 471/04; C07D 219/08; C07H 21/04; C08G 69/00

(52) U.S. Cl. 435/6; 546/70; 546/104; 536/23.1; 528/327

(57) ABSTRACT

The present invention relates to a polymer composed by two to ten monomers of formula (I) as well as to a process for its preparation and its use as fluorophore wherein: X is a radical of formula (II) wherein —R₅ is an electron pair or a (C₁-C₃)-alkyl radical; —Rₐ and —R_{b} are radicals independently selected from the group consisting of H, (C₁-C₄)-alKyI, (C₁-C₄)-alkoxy, (C₁-C₄)-alkylamino, phenyl, F, Cl, Br, amino, hydroxy, and nitro or —Rₐ and —R_{b} are fused forming with the carbon atoms to which they are attached a ring of formula (III) with the condition that (I) when —R₅ is an electron pair, a is a N═C double bond, and Rₐ and R_{b} are fused forming the ring (III), said ring being a biradical selected from (IIIa) and (IIIb), thus, radical (II) is (IIa) or (IIb) respectively (II) when —R₅ is a (C₁-C₃)-alkyl radical, a is a N—C single bond and Rₐ and R_{b} are fused forming the ring (III), said ring being a biradical (a), thus, the radical (II) is (IIc) R₁-R₄ and R₇-R₁₈ represent radicals, same or different, selected for the group consisting of H₁ (C₁-C₄)-alkyl, (C₁-C₄)-alky-lamiπo, phenyl, F, Cl, Br, amino, hydroxy, and nitro; p is an integer from 0 to 1; R₆ is a biradical selected from the group consisting of —CO—; —CONH(CH₂)ₘCO—; and —CO[NHCHR″CO]ₘ—, wherein —R″ are side chains radicals, same or different, corresponding to natural aminoacids; and m is an integer from 1 to 3; Z is a triradical of formula (IV) wherein r is an integer from 0 to 1; v is an integer from O to 1; Z′ is a triradical selected from —CH₂— and nitrogen; Z″ is H, with the proviso that: (a) when Z′ is nitrogen, forming an amide bound with R₆, then is hydrogen and v is an integer from O to 1, and (b) when Z″ is —NH—, forming an amide group with R₆, then Z1 is —CH₂ and v=O or of formula (V) wherein Z″ is selected from —CH₃ and —CH₂NH—, Z^{1v} is selected from H and NH, Z^{v} is selected from S and O atom, W is an integer from 0 to 1, with the proviso that (c) when R₆ is bound to Z′″ then Z′″ is —CH₂NH—, Z^{1v} is hydrogen and w is 0; and (d) when Z^{1v} is —NH— forming an amide bound with R₆, Z′″ is —CH₃ and w is 1; and and wherein the monomers of formula (I) are linked through the triradical Z, forming an amide or phosphate bound.

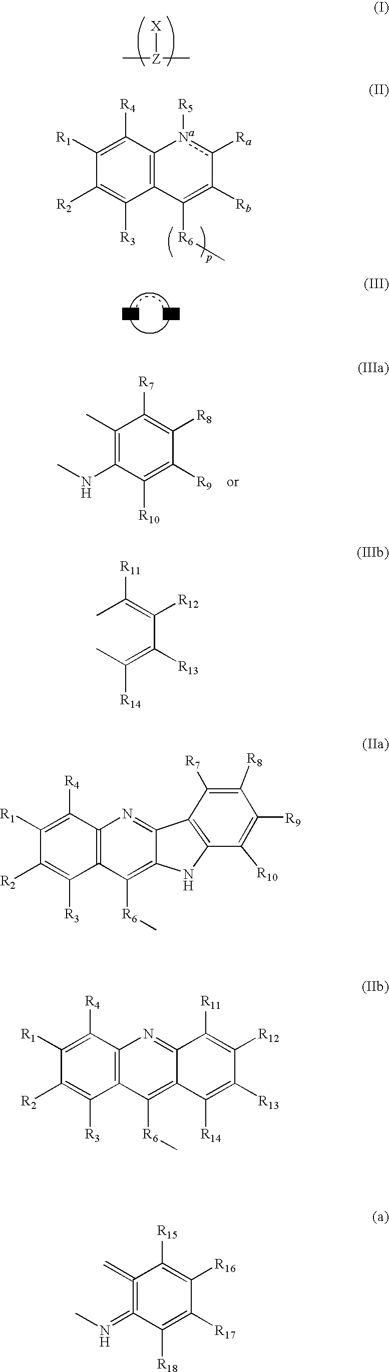

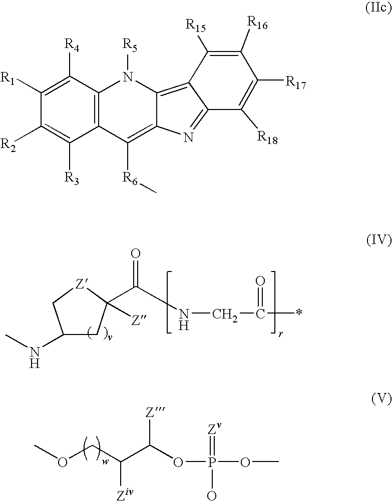

The present invention is related to the field of chemistry and molecular biology investigation. More particularly, the present invention refers to synthetic polymers which owing to their fluorescence properties are useful as labels of biological material.

### BACKGROUND ART

Fluorescence is the result of a three-stage process that occurs in certain molecules (generally polyaromatic hydrocarbons or heterocycles) named fluorophores or fluorescent dyes. In this process a photon supplied by an external source is absorbed by the fluorophore creating an excited electronic state. The excited state exists for a finite time while the fluorophore undergoes conformational changes, interacting with its molecular environment and dissipating energy yielding a relaxed excited state from which fluorescence emission originates. In the last step a photon is emitted returning the fluorophore to its ground state. Due to energy dissipation during the excited-state lifetime, the energy of this photon is lower and therefore of longer wavelength than the excitation photon.

Fluorescent based techniques are powerful tools for the investigation of biological material. The process of fluorescence emission occurs in a time scale between nanoseconds and milliseconds, dependent on the fluorescence system used. Since in this time scale many dynamic events take place, fluorescence can provide information on the structure, mobility, macromolecular size, distances or conformational rearrangements of dye-bound molecules.

In addition, fluorophores are becoming increasingly useful in combinatorial chemistry and biology, both as encoders of library members and as reporters of chemical reactions. Fluorescent nucleic acids in particular are important tools in molecular biology, diagnostics and structural studies. In order to make oligonucleotides fluorescent, a wide range of chemical and enzymatic methods were developed.

As a result of the greatly expanding use of fluorescent labels in research and diagnostic applications, there is a corresponding increase in the need for new fluorophores having a wider range of spectral characteristics along with improved properties.

### SUMMARY OF THE INVENTION

Inventors have found that the polymers according to the present invention have fluorescent properties. Particularly, when they are used as fluorophores they may be attached to molecules that are not fluorescent and the resulting molecule acquires the fluorescent properties of the fluorphore. It has been found, furthermore, that the attachment of the compounds of the present invention to the molecules to be tested is specific. This specific attachment confers high sensitivity and selectivity to the fluorescent based technique when the compounds of the invention are used as fluorophores or fluorescence labels. The main advantage of the improvement in sensitivity and selectivity is that a minor amount of the sample to be analyzed is needed, avoiding the inconveniences associated with the acquisition and processing of tissue samples.

It is known in the state of the art that when fluorophores are used, they may exert an undesirable influence on the structure and mobility of the sample. These changes in the conformation of the sample can lead to a decreased specificity.

As it is illustrated below, the compounds of the present invention attaches to a target molecule (e.g., an oligonucleotide) being preserved the structure of the target. Furthermore, the inventors of the present invention have shown that the compounds when attach to the specific molecule increase the stability of said molecule (as it is derived from the melting temperature data).

Thus, a first aspect of the present invention is a polymer composed by two to ten monomers of formula (I)

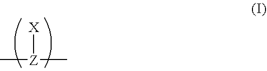

wherein:
X is a radical of formula (II)

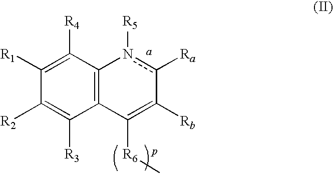

wherein
—R₅ is an electron pair or a (C₁-C₃)-alkyl radical;
—Rₐ and —R_{b} are radicals independently selected from the group consisting of H, (C₁-C₄)-alkyl, (C₁-C₄)-alkoxy, (C₁-C₄)-alkylamino, phenyl, F, Cl, Br, amino, hydroxy, and nitro; or
—Rₐ and —R_{b} are fused forming with the carbon atoms to which they are attached a ring of formula (III)

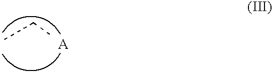

with the condition that
(i) when —R₅ is an electron pair, a is a N═C double bond, and Rₐ and R_{b} are fused forming the ring

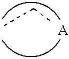

said ring being a biradical selected from (IIIa) and (IIIb)

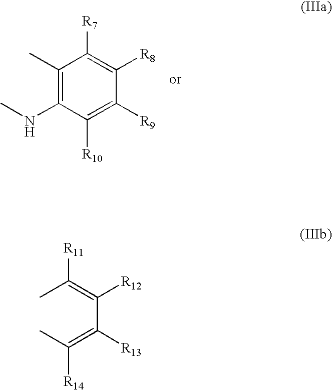

thus, radical (II) is (IIa) or (IIb) respectively

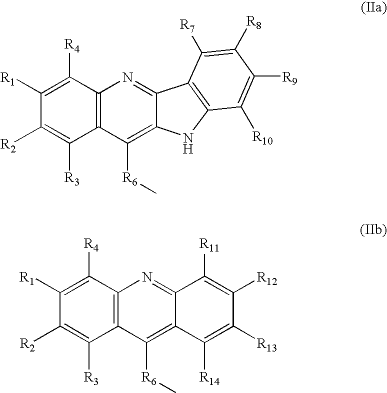

(ii) when —R₅ is a (C₁-C₃)-alkyl radical, a is a N—C single bond and Rₐ and R_{b} are fused forming the ring
said ring being a biradical

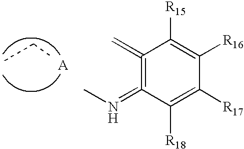

thus, the radical (II) is (IIc);

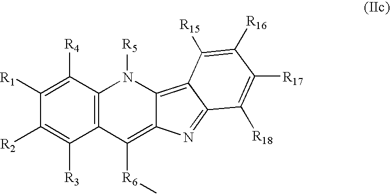

R₁-R₄ and R₇-R₁₈ represent radicals, same or different, selected from the group consisting of H, (C₁-C₄)-alkyl, (C₁-C₄)-alkylamino, phenyl, F, Cl, Br, amino, hydroxy, and nitro;
   p is an integer from 0 to 1;

R₆ is a biradical selected from the group consisting of —CO—; —CONH(CH₂)ₘCO—; and —CO[NHCHR″CO]ₘ—, wherein —R″ are side chains radicals, same or different, corresponding to natural aminoacids; and m is an integer from 1 to 3;

Z is a triradical of formula (IV)

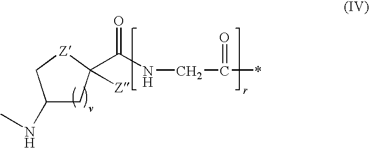

wherein
   r is an integer from 0 to 1;
   v is an integer from 0 to 1;
   Z′ is a triradical selected from —CH₂— and nitrogen;
   Z″ is H,
with the proviso that:
   (a) when Z′ is nitrogen, forming an amide bound with R₆, then Z″ is hydrogen and v is an integer from 0 to 1, and
   (b) when Z″ is —NH—, forming an amide group with R₆, then Z′ is —CH₂— and v=0,
      or of formula (V):

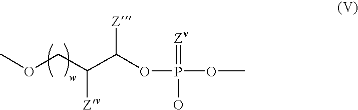

wherein
   Z′″ is selected from —CH₃ and —CH₂NH—,
   Z^{iv} is selected from H and NH,
   Z^{v} is selected from S and O atom,
   W is an integer from 0 to 1,
with the proviso that
   (c) when R₆ is bound to Z′″, then Z′″ is —CHNH—, Z^{iv} is hydrogen and w is 0; and
   (d) when Z^{iv} is —NH— forming an amide bound with R₆, Z′″ is —CH₃ and w is 1; and
      and wherein the monomers of formula (I) are linked through the triradical Z, forming an amide or phosphate bound.

It is believed that the invention polymer's fluorescence properties previously mentioned are due to the nature of the monomers composing said polymer. The X radical of the monomers composing the polymers has as central structure a ring system comprising two aromatic rings fused, one of said aromatic rings having a nitrogen atom as heteroatom. Said ring system confers to the polymers of the invention the fluorescence properties. On the other hand, the Z radical (i.e. the backbone) used to connect the fluorescent units (i.e. “X” radical) plays the role of linking the monomers, not affecting to the binding ability of the polymer to the target sequence. In fact, as shown below, there is a high affinity for multistranded nucleic acid sequences that have a more dense negative charge (triplex and G-quadruplex). G-Quadruplex nucleic acid structures are biologically-relevant structures. These structures are found at the end of the chromosomes (telomeres) to maintain chromosome integrity as well as throughout the human genome. They are especially abundant at the promoter regions of oncogenes. Compounds that bind these sequences may help understanding the functional role of these sequences. This is of great importance in the diagnostic and prognostic fields wherein the identification of a specific sequence is needed to determine in an accurate way, for instance, the risk to develop an illness, if the subject has already developed it or how the condition will go on.

A second aspect of the present invention is a process for preparing a polymer according to the first aspect of the invention which is carried out coupling compounds of formula (VI):

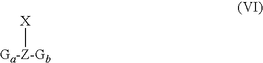

wherein
X and Z are as defined in above, and
Gₐ and G_{b} are protecting groups,
   on a solid-phase support.

A third aspect of the present invention is compound of formula (VI):

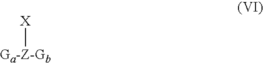

wherein X, Z, Gₐ and G_{b} are as defined above.

In a fourth aspect, the present invention relates to the use of a polymer according to the first aspect of the invention as a fluorophore.

Illustrative non-limitative examples of the applications of the polymers according to the first aspect of the invention as fluorophores are the following:
Automated sequencing of DNA by the chain termination method; each of four different chain terminating bases has its own specific fluorescent tag. As the labelled DNA molecules are separated, the fluorescent label is excited by a UV source, and the identity of the base terminating the molecule is identified by the wavelength of the emitted light.
DNA detection for visualising the location of DNA fragments;
The DNA microarray;
Immunology: An antibody has a fluorescent chemical group attached, and the sites (e.g. on a microscopic specimen) where the antibody has bound can be seen, and even quantitated, by the fluorescence.
FACS (fluorescent-activated cell sorting)
The study of the structure and conformations of DNA and proteins. This is especially important in complexes of multiple biomolecules.

Fluorescence techniques using fluorescent labeled DNA probes have the potential for developing homogeneous, relatively inexpensive, and easy to use DNA probe assays, due to the sensitivity of the emission intensity of the fluorescent label to environmental changes. Such assays are possible if the hybridization of the probe with the target sequence is accompanied by a change in one or more fluorescent properties such as fluorescence quantum yield, lifetime, polarized emission, fluorescence quenching, excitation transfer or sensitized fluorescence. The target sequence can thus be detected and quantified from the change in properties occurring when the target sequence is added to an analysis tube containing the appropriate DNA probe. Thus, in a fifth aspect the present invention provides a method of preparing a fluorescently labeled nucleic acid molecule which comprises incorporating at least one polymer according to the first aspect of the invention into a RNA or DNA molecule under conditions sufficient to incorporate said polymer.

In a sixth aspect, the present invention provides a method of detecting a target nucleic acid in a sample to be tested which comprises contacting the target nucleic acid with a nucleic acid probe comprising at least one polymer according to the first aspect of the invention, for a time under conditions sufficient to permit hybridization between said target and said probe; and detecting said hybridization.

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention

### DESCRIPTION OF DRAWINGS

FIG. 1 shows the specificity of polymers to specific DNA sequences. This specificity is determined by competitive dialysis experiment of the dimers Aca-p-Aca (black bar), QgpQgp (white bar), and the hexamer Act-p-Act-p-Act-p-Act-p-Act-p-Act (grey bar) for certain sequences. The amount shown in the y axis is the concentration of the polymer.

FIG. 2 shows the specificity of polymers to specific DNA sequences. This specificity is determined by competitive dialysis experiment of the following trimers of the invention: Act-p-Qut-p-Agt (black bar), Act-p-Qut-p-Act (grey bar), Act-p-Qut-p-Nct (white bar), Qut-p-Qut-p-Qut (dotted bar), Act-p-Qut-p-Qut (scuared bar), Act-p-Act-p-Act (scratched bar) for certain sequences. The amount shown in the y axis is the concentration of the polymer.

FIG. 3 shows the specificity of polymers to specific DNA sequences. This specificity is determined by competitive dialysis experiment of dimmers Act-p-Act (white bar) and Act-p-Qct (black bar) for certain quadruplex sequences. The amount shown in the y axis is the concentration of the polymer.

### DETAILED DESCRIPTION OF PARTICULAR EMBODIMENTS

In a particular embodiment of the present invention, the X radical of formula (II) comprises a ring system containing at least two fused aromatic rings, preferably between 2 and 4 fused aromatic rings.

In one embodiment of the first aspect of the invention, the triradical Z group corresponds to the formula (IV). Preferably is selected from the group consisting of:

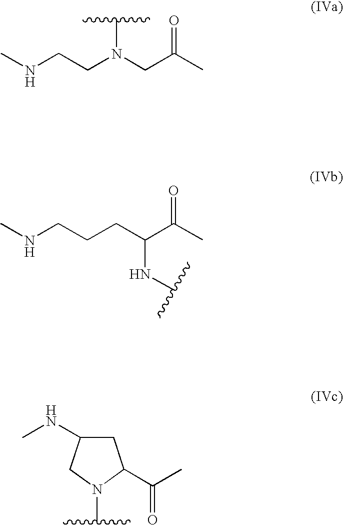

wherein the symbol [symbol] indicates the position through which radical X is attached by its biradical R₆.

In another embodiment of the first aspect of the invention, the triradical Z group corresponds to the formula (V). Preferably the triradical Z of formula (V) is selected from the group consisting of:

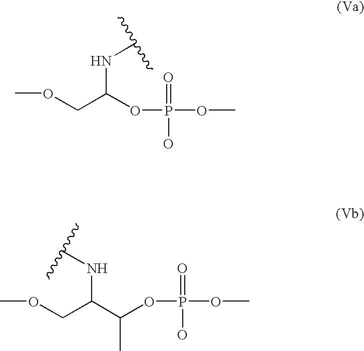

wherein the symbol [symbol] indicates the position through which triradical Z is attached to the radical X.

In another embodiment of the first aspect of the invention, the radical X of formula (II) is selected from the group consisting of:

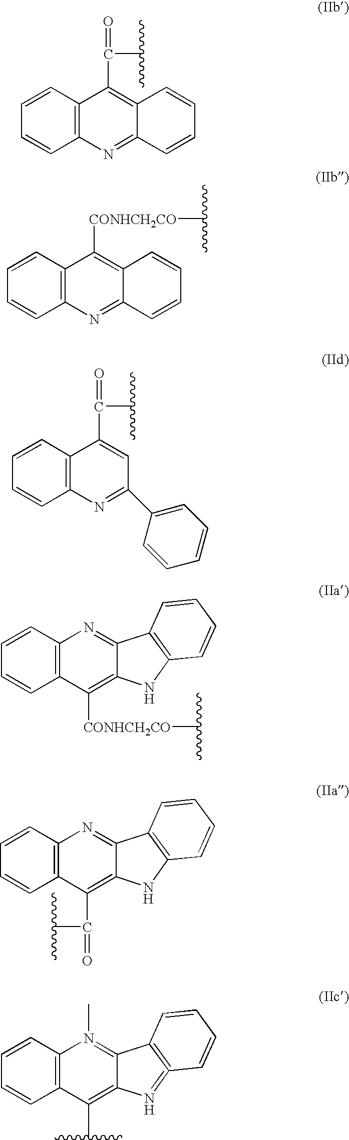

wherein the symbol [symbol] indicates the position through which radical X is attached by its radical R₆ to the triradical Z.

In still another embodiment, R₆ is —CO[NHCHR″CO]ₘ—, being —R″ the side chain radical corresponding to the aminoacid selected from glicine and proline; and m is an integer from 1 to 3.

Preferably the polymer is a homopolymer.

In the present invention, the term “homopolymer” is to be understood as that which is constructed of identical monomers of formula (I) as defined above.

In another embodiment of the first aspect of the invention the polymer is a heteropolymer.

In the present invention, the term “heteropolymer” is to be understood as a polymer which is composed by different monomers of formula (I).

The polymer according to the present invention is selected from the group consisting of:

O-{2-N-(Acridine-9-carbamoyl)-(1-hydroxybut-3-yl)} and O-{2-N-(10H-indolo[3,2-d]quinoline-11-carbamoyl)-3-hydroxybut-1-yl} phosphate;

O-{2-N-(10H-indolo[3,2-d]quinoline-11-carbamoyl)-(1-hydroxybut-3-yl)} and O-(2-N-{10H-indolo[3,2-d]quinoline-11-carbamoyl}-3-hydroxybut-1-yl)phosphate;

-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-(2-N-{10H-indolo[3,2-d]quinoline-11-carbamoyl}-3-hydroxybut-1-yl);

O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-3-hydroxybut-1-yl};

O-{2-N-(Acridine-9-carbamoyl)-(1-hydroxybut-3-yl)} and O-{3-(5-methyl-5H-indolo[3,2b]quinolin-11-ylamino-(S)-(2-hydroxyprop-3-yl} phosphate;

O-{2-N-(Acridine-9-carbamoyl)-(1-hydroxybut-3-yl)} and O-{3-(5-methyl-5H-indolo[3,2b]quinolin-11-ylamino-(S)-(2-hydroxyprop-3-yl} phosphorotioate;

O-[(R)-3-(Acridin-9-ylamino)-1-hydroxy-propane-2-yl]-phosphate (2-1) O-{2-N-(acridine-9-ylamino)-2-oxyprop-1-yl} phosphate(2-4) 4-hydroxybutiramide;

O-{2-N-(2-phenylquinoline-4-carbamoyl-1-hydroxybutan-3-yl)} and O-{2-N-(acridine-9-carbamoyl)-3-hydroxybut-1-yl} phosphate;

O-{2-N-(2-phenylquinoline-4-carbamoyl-1-hydroxybutan-3-yl)} and O-{2-N-(2-N-(2-phenylquinoline-4-carbamoyl-3-hydroxybutan-1-yl)} phosphate;

O-{2-N-(acridine-9-carbamoyl)-1-hydroxybut-3-yl} and O-{2-N-(2-phenylquinoline-4-carbamoyl-3-hydroxybutan-1-yl)} phosphate;

O-{2-N-(2-phenylquinoline-4-carbamoyl-1-hydroxybutan-3-yl)} and O-(2-N-{10H-indolo[3,2-d]quinoline-11-carbamoyl}-3-hydroxybut-1-yl);

O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carboxamido)acetamido]-(3-hydroxybut-1-yl)};

(2S, 3R)-2-((9H-Fluoren-9-yl)methyloxycarbonyl)amino)butane-1-(4,4′-dimethoxytrityloxy)butan-3-ol;

(2S, 3R)-2-((9H-Fluoren-9-yl)methyloxycarbonyl)amino)butane-1-(4,4′-dimethoxytrityloxy)butan-3-yl N,N-diisopropylamino-2-cyanoethyl phosphoramidite;

O-{2-N-(10H-indolo[3,2-d]quinoline-11-carbamoyl)-3-oxybut-1-yl} phosphate (3-1) O-{3-(5-methyl-5H-indolo[3,2-b]quinolin-11-ylamino-(S)-(2hydroxylprop-3-yl);

O-{2-N-(10H-indolo[3,2-d]quinoline-11-carbamoyl)-3-oxybut-1-yl} phosphate (3-1) O-{2-N-(10H-indolo[3,2-d]quinoline-11-carbamoyl)-3-oxybut-1-yl} phosphate (3-1) O-(2-N-{10H-indolo[3,2-d]quinoline-11-carbamoyl}-3-hydroxybut-1-yl);

O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(3-hydroxybut-1-yl)};

O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carbamoyl)-(3-hydroxybut-1-yl)};

O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(5H-indolo[2,3-b]quinolin-5-yl)acetamido-3-hydroxybut-1-yl);

Acetyl-{2-[Acridine-9-carbonylyamino]-acetyl}-(2-aminoethyl)-glycyl-{2-[Acridine-9-carbonylyamino]-acetyl}-(2-aminoethyl)-N-6-hydroxyhexyl glycynamide;

Acetyl-{2-[Acridine-9-carbonylyamino]-acetyl}-(2-aminoethyl)-glycyl-{2-[Acridine-9-carbonylyamino]-acetyl}-(2-aminoethyl)-glycyl {2-[Acridine-9-carbonyl)-amino]-acetyl}-(2-aminoethyl)-N-6-hydroxyhexyl glycynamide;

Acetyl-{2-N-(10H-indolo[3,2-d]quinoline-11-carbonyl)-amino]-acetyl}-(2-aminoethyl)-glycyl-{2-N-(10H-indolo[3,2-d]quinoline-11-carbonyl)-amino]-acetyl}-(2-aminoethyl)-N-6-hydroxyhexyl glycynamide;

N-[2-(Acridine-9-carboxamide)]-4-N-[2-(acridine-9-carboxamide)prolinamide;

N-[2-(Acridine-9-carboxamide)acetyl]-4-N-acetamidoprolinamide;

N-{2-(10H-indolo[3,2-b]quinoline-11-carboxamide)acetyl}-4-N-acetamidoprolinamide;

N⁴-Acetyl-4-amino-{2-(acridine-9-carboxamide)acetyl}prolinyl-4-amino-{2-(acridine-9-carboxamide)acetyl}prolinamide;

N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinamide;

10H-Indolo[3,2-b]quinoline-11-carboxylic acid {2-[4-acetylamino-2-(1-{2-[(acridine-9-carbonyl)-amino]-acetyl}-5-carbamoyl-pyrrolidin-3-ylcarbamoyl)-pyrrolidin-1-yl]-2-oxo-ethyl}-amide;

10H-Indolo[3,2-b]quinoline-11-carboxylic acid (2-{4-[(4-acetylamino-1-{2-[(acridine-9-carbonyl)-amino]-acetyl}-pyrrolidine-2-carbonyl)-amino]-2-carbamoyl-pyrrolidin-1-yl}-2-oxo-ethyl)-amide;

N⁴-Acetyl-4-amino-{2-(acridine-9-carboxamide)acetyl}prolinyl-4-amino-{2-(acridine-9-carboxamide)acetyl}prolinyl-4-amino-{2-(acridine-9-carboxamide)acetyl}prolinamide;

N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-{2-(10H-indolo[3,2-b]quinoline-11-carboxamide)acetyl}prolinamide;

N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(acridine-9-carboxamide)acetyl}prolinamide;

N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-4-amino-{2-(acridine-9-carboxamide)acetyl}prolinamide;

N⁴-Acetyl-4-amino-{2-(10H-indolo[3,2-b]quinoline-11-carboxamide)acetyl}prolinyl-4-amino-{2-(acridine-9-carboxamide)acetyl}prolinyl-4-amino-{2-(acridine-9-carboxamide)acetyl}prolinamide;

N⁴-Acetyl-4-amino-{2-(10H-indolo[3,2-b]quinoline-11-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide;

N⁴-Acetyl-4-amino-{2-(acridine-9-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide;

N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(10H-indolo[3,2-b]quinoline-11-carboxamide)acetyl}prolinamide;

N⁴-Acetyl-4-amino-{2-(10H-indolo[3,2-b]quinoline-11-carboxamide)acetyl}prolinyl-4-aminoprolinyl-4-amino-{2-(acridine-9-carboxamide)acetyl}prolinyl-4-aminoprolinyl-4-amino-[(2-phenyl-quinoline-4-carboxamide)acetyl]prolinamide;

N⁴-Acetyl-4-amino-{2-(acridine-9-carboxamide)acetyl}prolinyl-4-aminoprolinyl-4-amino-[(2-phenyl-quinoline-4-carboxamide)acetyl]prolinamide-4-aminoprolinyl-4-amino-{2-(10H-indolo[3,2-b]quinoline-11-carboxamide)acetyl}prolinamide;

N⁴-Acetyl-4-amino-{2-(10H-indolo[3,2-b]quinoline-11-carboxamide)acetyl}prolinyl-4-aminoprolinyl-4-aminoprolinyl-4-amino-{2-(acridine-9-carboxamide)acetyl}prolinyl-4-aminoprolinyl-4-aminoprolinyl-4-amino-[(2-phenyl-quinoline-4-carboxamide)acetyl]prolinamide;

N⁴-Acetyl-4-amino-{2-(acridine-9-carboxamide)acetyl}prolinyl-4-aminoprolinyl-4-aminoprolinyl-4-amino-[(2-phenyl-quinoline-4-carboxamide)acetyl]prolinamide-4-aminoprolinyl-4-aminoprolinyl-4-amino-{2-(10H-indolo[3,2-b]quinoline-11-carboxamide)acetyl}prolinamide;

N⁴-Acetyl-5-N-{2-(acridine-9-carboxamide)acetyl}ornithinyl-5-N-{2-(acridine-9-carboxamide)acetyl}ornithinyl-5-N-{2-(acridine-9-carboxamide)acetyl}ornithinamide;

N⁴-Acetyl-5-N-{2-(acridine-9-carboxamide)acetyl}ornithinyl-5-N-{2-(10H-indolo[3,2-b]quinoline-11-carboxamide)acetyl}ornithinyl-5-N-{2-(acridine-9-carboxamide)acetyl}ornithinamide;

N⁴-Acetyl-5-N-{2-(acridine-9-carboxamide)acetyl}ornithinyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinamide; and

N⁴-Acetyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinamide.

“Protecting group” as used in the present invention is a group that is attached to, or placed on, an atom so the protected atom does not react with reactants, thereby temporarily rendering the protected atom inactive. Illustrative examples of protecting groups are tetrahydropyran (THP), t-butyloxy carbonyl (BOC) and fluoromethyloxy carbonyl (FMOC). A comprehensive list and description of protecting groups can be found in Protective groups in Organic Synthesis, second edition, T. W. Greene and P.G.M. Wuts, 1991, which are incorporated herein by reference.

In one embodiment of the second aspect of the invention, Gₐ and G_{b} are selected from dimethoxytrityl, —P(OCH₂CH₂CN)—N(isopropyl)₂ and t-butoxy carbonyl protecting groups.

In one embodiment of the third aspect of the invention, the compound is linked to a solid support through one of the protecting groups Gₐ and G_{b}.

### Examples

I. Synthesis of the Compounds of Formula (VI).

A group of preferred compounds of formula (VI) are those derived from threoninol and which corresponds to formula (VIa).

Threoninol derivatives (VIa), wherein R₁-R₅, Rₐ, R_{b} and m are as defined above, can be prepared according to the process summarized in Scheme 1.

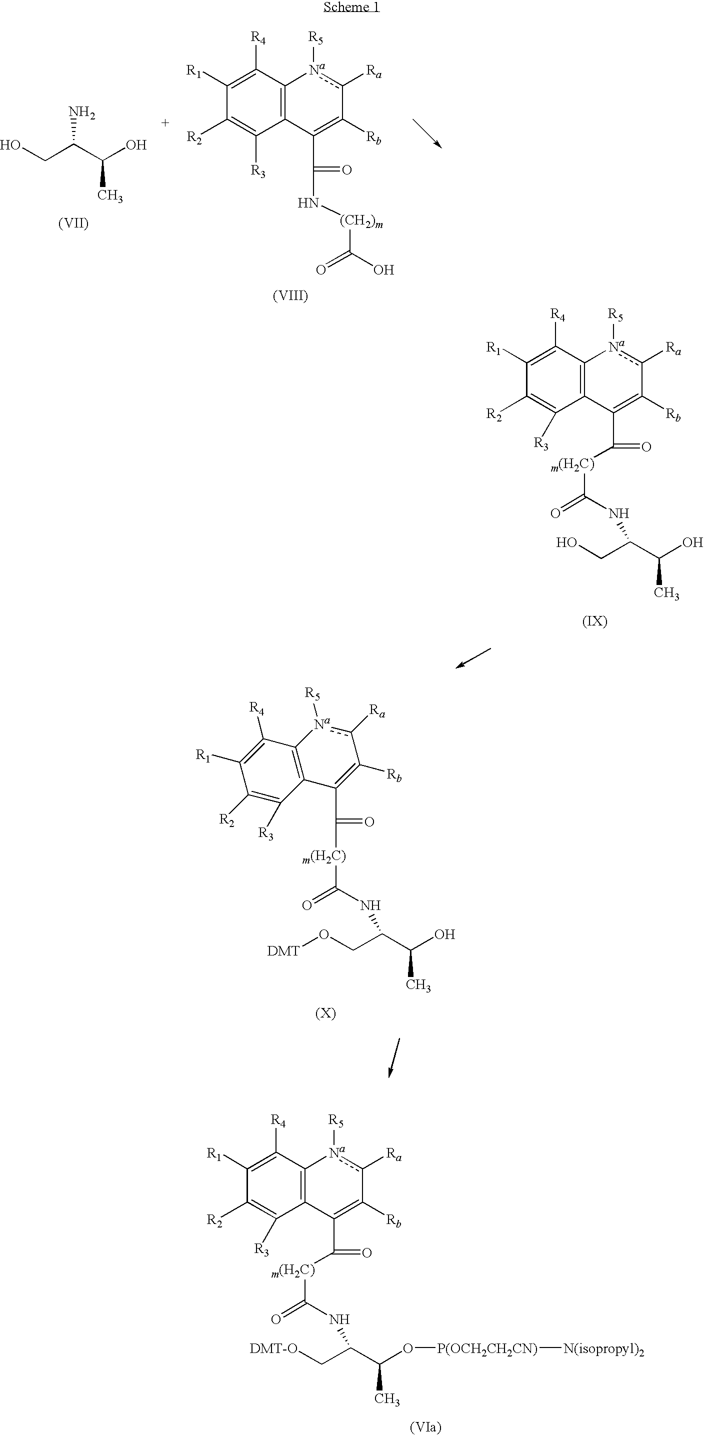

L-Threoninol (VII) was reacted with carboxylic derivatives (VIII) using diisopropylcarbodiimide and 1-hydroxybenzotriazole yielding compounds of formula (IX). In order to increase the distance between the compound of formula (VIII) and the threoninol molecule (VII) for those cases in which m is different from 0, a glycine is added by reaction of the carboxyl derivative of the compound with glycine methyl ester and subsequent hydrolysis of the methyl ester to yield derivatives VI. The compounds (IX) are reacted with dimethoxytrityl chloride in pyridine to yield 4,4′-dimethoxytrityl (DMT) derivatives (X) that were reacted with chloro-N,N-diisopropylamino-O-2-cyanoethoxy phosphine to yield phosphoramidites of formula (VIa).

Another group of preferred compounds are those derived from 3-amino-1,2-propandiol and which corresponds to formula (VIb).

The synthesis of the 3-amino-1,2-propandiol derivatives of formula (VIb) is summarized in Scheme 2:

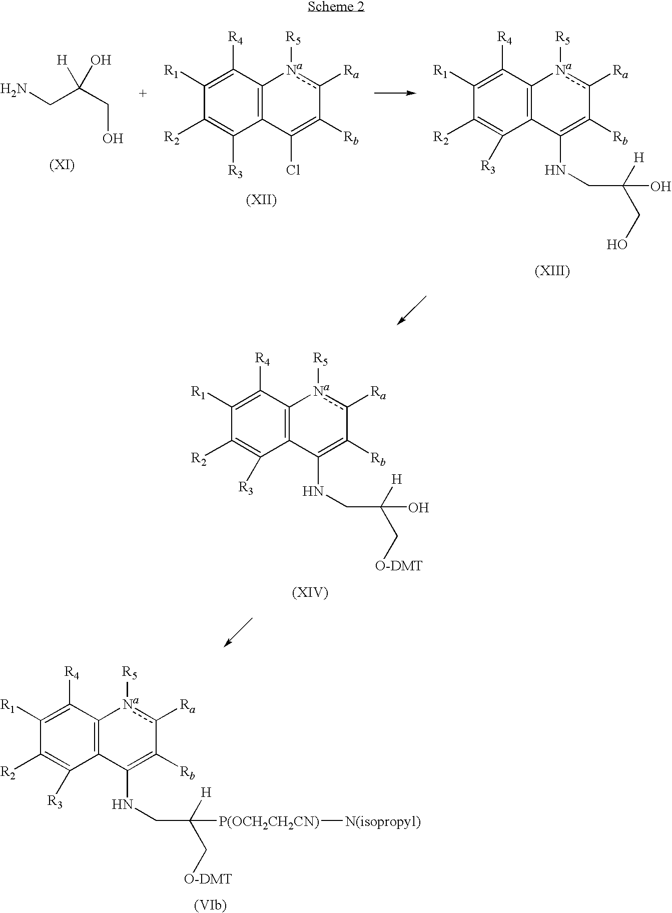

The R-isomer or the S-isomer of 3-amino-1,2-propandiol (XI) were reacted with the chloro derivative of formula (XII) yielding the corresponding diol derivatives (XIII). The resulting compounds were reacted with dimethoxytrityl chloride in pyridine to yield DMT derivatives (XIV) followed by reaction with chloro-N,N-diisopropylamino-O-2-cyanoethoxy phosphine to yield phosphoramidites (VIb).

Another group of preferred compounds are those derived from 2-aminoethylglycine derivatives which corresponds to formula (VIc).

The synthesis of 2-aminoethylglycine derivatives of of formula (VIc) is summarized in scheme 3.

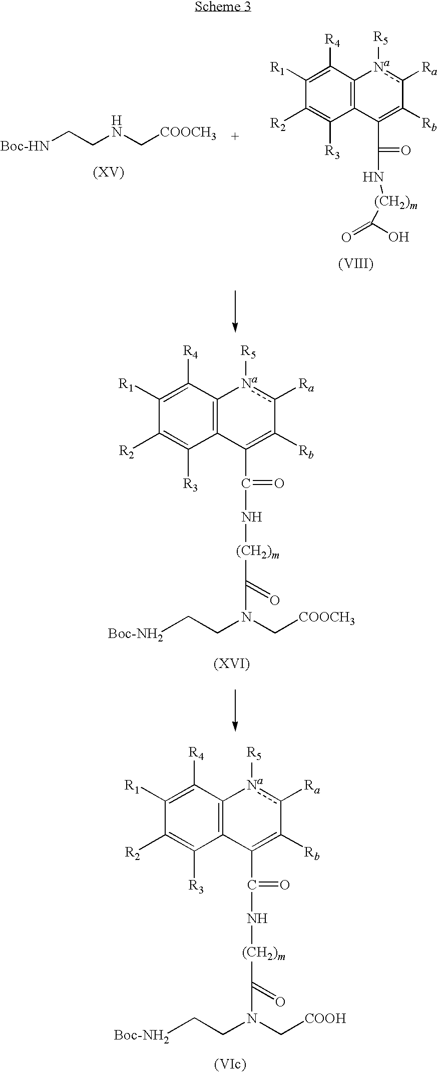

The N-Boc-protected derivative of 2-aminoethylglycine methyl ester (XV) is reacted with carboxyl derivative (VIII) using diisopropylcarbodiimide and 1-hydroxybenzotriazole yielding compound (XVI). Hydrolysis of the resulting compound with aq. NaOH in dioxane (1:1) gave the desired derivative (VIc) in good yields.

II. Synthesis of Polymers

In order to synthesize polymers using solid-phase protocols, DMT derivatives of DNA-intercalators described above were coupled to controlled pore glass supports using the succinyl linker as described in the bibliography. For this purpose DMT-derivatives described above were reacted with succinic anhydride followed by coupling of the resulting hemisuccinates with amino functionalized controlled pore glass yielding glass beads loaded with the appropriate intercalating compounds (c.f. R. T. Pon “Solid-phase supports for oligonucleotide synthesis” in Methods in Molecular Biology Vol. 20: Protocols for oligonucleotides and analogs, Edited by S. Agrawal, Humana Press Inc., Totowa, N.J., USA (1993), pp 465-496).

Phosphoramidites described above were assembled into dimeric and trimeric strands using the appropriate solid supports (1-4 μmol scale). Standard phosphoramidite chemistry was used (c.f M. H. Caruthers et al., “Chemical synthesis of deoxyoligonucleotides by the phosphoramidite method” Methods Enzymol., 1987, vol. 154, pp 287-313). This consist in cycles with four chemical reactions: 1) removal of the dimethoxytrityl group with 3% trichloroacetic acid in dichloromethane; 2) phosphoramidite coupling using 4-5 times excess of phosphoramidite and 20 times excess of tetrazole; 3) capping with acetic anhydride and N-methylimidazole and 4) phosphite oxidation to phosphate with 0.01 M iodine in tetrhydrofurane/pyridine/water. Coupling yields were between 80-95%.

After the assembly of the sequences, supports were treated with conc. aqueous ammonia for 1-16 hrs at 20-60° C. to yield the desired unprotected oligomers. Excellent yields where obtained when oligomers contain L-threoninol backbone.

Some polymers with the propan-1,2-diol backbone were not stable to hot ammonia solutions. Alternatively these compounds were deprotected either with 1) a 0.5 M 1,8-diazabicyclo[5.4.0]undec-7-ene (DBU) solution in pyridine, and b) NH₃ in methanol at room temperature for 2-4 hr, but yields were very low. Best results were obtained using a new strategy based on an acid labile linker. In this strategy 4-trityl-4-hydroxybutanoic acid was reacted with polystyrene loaded with Rink-amide linker (c.f. H. Rink, “Solid-phase synthesis of protected peptide fragments using a trialkoxy-diphenyl-methylester resin” Tetrahedron Lett., 1987, vol 28, pp 3787). The resulting support was used for the assembly of the s as described above. After the assembly of the sequences, the supports were treated with 0.5 M DBU solution for 1 min, washed with acetonitrile and treated with a solution containing 95% trifluoroacetic acid and 5% water for 4 hr at room temperature. Using this conditions oligomers with the propan-1,2-diol backbone were obtained in good yields.

Polymers with N-am inoethylglycine backbone were synthesized on polyethyleneglycol-polystyrene supports with the 6-aminohexylsuccinyl linker (c.f. D. W. Will et al., “The synthesis of polyamide nucleic acids using a monomethoxytrityl protecting-group strategy” Tetrahedron, 1995, vol. 51, pp 12069-12082). Standard solid-phase peptide synthesis protocols were used. The synthesis cycle consist of two reactions: 1) Deprotection of Boc with a 5% cresol solution in 95% trifluoroacetic acid and 2) coupling of the appropriate monomer (5 molar excess) HATU (4.5 molar excess) and N,N-diisopropylethylamine (15 molar excess). After the assembly of the sequence, the resulting supports were treated with conc. aqueous ammonia for 2 hrs at 55° C. to yield the desired unprotected oligomers.

In another protocol, a phosphoramidite derivative of threoninol was prepared. In this derivative, the amino group was protected with the Fmoc group and the primary alcohol function with the DMT group. The phosphoramidite group was incorporated in the secondary alcohol. This phosphoramidite was incorporated into a sequence using standard oligonucleotide synthesis protocols (example 60, 1-Qut-p-Cra-2). Removal of the Fmoc group with a 20% piperidine solution yields a free amino group that may be further derivatized with a drug carrying a carboxylic acid function. This protocol is known as submonomeric approach and it has been also used for the synthesis of PNA derivatives.

Polymers with the 4-aminoproline were assembled to the MBHA resin applying an Fmoc/Boc hybrid strategy and using trans-γ-Fmoc-amino-α-Boc-L-proline [Boc-Amp(Fmoc)-OH] as a amino acid (c.f. J. Farrera-Sinfreu et al., “A new class of foldamers based on cis-γ-amino-L-proline” JAGS, 2004, 126, 6048-6057). Fmoc was the temporary protecting group for the γ-amino functionality of each monomer (for the backbone elongation thus). Boc was the semi-permanent protecting group for the α-amino group through which the fluorophores were introduced. α Amino derivatizations were carried out on solid-phase using standard Boc chemistry protocols. At this α-amino position, the addition of a glycine molecule as spacer allowed the intercalator introduction. In general, Fmoc group was removed using 20% of piperidine in DMF and Boc-Amp(Fmoc)-OH (5 eq) was coupled to the resin using DIPCDI (5 eq) and HOBt (5 eq) as coupling reagents. Boc groups were removed using 40% TFA in DCM, followed by the resin treatment with 5% DIEA in DCM. Boc-Gly-OH (5 eq) was coupled to the resin using DIPCDI (5 eq) and HOBt (5 eq) as coupling reagents. Intercalators (3 eq) were coupled to the resin using TBTU (3 eq) and DIEA (6 eq) as coupling reagents. Acetylations were carried out using Ac₂O (5 eq) and DIEA (5 eq).

Thus, the synthesis of these polymers were carried out following different solid-phase procedures depending on the nature of the final oligomers (homopolymers or heteropolymers).

Protocol I. This protocol was applied for those polymers wherein the monomers were the same and introduced at the α-positions (homopolymers). Thus, the γ-peptidic backbone was synthesized first by repetitive couplings of Boc-Amp(Fmoc)-OH using Fmoc chemistry and, after removal of all Boc protecting groups, the spacer (Boc-Gly-OH) was introduced at the α-amino positions. After the Boc groups removal, fluorophores were introduced through these glycines, forming the final homopolymers. In this way, different dimers, trimers and tetramers were synthesised.

Protocol II. This protocol was applied for those polymers that have different monomers at each α-amino position of the different prolines (heteropolymers). In this procedure, the monomer was introduced in a sequential way after each aminoproline coupling. Thus, Boc-Amp(Fmoc)-OH was coupled to the MBHA resin, the Boc group was removed and the spacer (Boc-Gly-OH) was coupled, the Boc group was removed and the monomer was introduced, obtaining the first aminoproline functionalised with the first fluorophore. Then, the Fmoc group was removed, the second aminoproline monomer was introduced, the Boc group was removed, the spacer (Boc-Gly-OH) was coupled at this α-amino position, the Boc group was removed and the second fluorophore introduced, obtaining an aminoproline dimer with two different monomers. Repetitions of these steps allowed the synthesis of several dimers, trimers and tetramers.

Protocol III. This protocol was applied for those polymers that did not have monomer at all the α-amino proline positions. These compounds were treated as heteropolymers. They were synthesised using protocol II, with the difference that those α-amino positions of the proline that did not have monomer were acetylated. Thus, the α-amino positions that were maintained without monomer were acetylated after the Boc group removal.

All γ-aminoproline oligomers were finally treated with 20% piperidine solution to remove the N-terminal Fmoc group, acetylated at this position and treated with anhydrous HF to obtain the desired products.

Ornitine polymers were synthesised following the same synthetic strategies used in the synthesis of γ-aminoproline oligomers, just replacing the scaffold. Backbone elongation was carried out through the δ-amino function using Fmoc chemistry and monomers were introduced in the α-amino group using Boc chemistry and using glycine as a spacer.

### Examples

### Example 1

### Preparation of 2-(10H-indolo[3,2-b]quinoline-11-carboxamide)acetic acid (Compound 1c)

10H-indolo[3,2-d]quinoline-11-carboxylic acid (Compound 1a) previously prepared (c.f. D. E. Bierer et al., “Ethnobotanical-directed discovery of the antihyperglycemic properties of cryptolepine: its isolation from Cryptolepis sanguinolenta, synthesis and in vitro and in vivo activities” J. Med. Chem. 1998, vol. 41, pp 894-901) (0.43 g, 1.64 mmol) was dissolved in 20 ml of dimethylformamide together with N,N-diisopropylcarbodiimide (0.25 ml, 1.64 mmol) and 1-hydroxybenzotriazole (0.22 g, 1.64 mmol). The mixture was stirred for 15 minutes. To this solution a mixture of glycine methyl ester hydrochloride (0.15 mg, 1.64 mmol) and N,N-diisopropylethylamine (0.28 ml, 1.64 mmol) dissolved in dimethylformamide was added. After 2 hrs of magnetic stirring at room temperature N,N-diisopropylethylamine (0.2 ml, 1.12 mmol) were added and stirring was continued for 1 hour. The resulting mixture was concentrated to dryness and the residue was dissolved in CH₂Cl₂. A precipitate was formed that was collected yielding 2-(10H-indolo[3,2-b]quinoline-11-carboxamide)acetic methyl ester (0.31, 57%). The organic phase was washed with 5% sodium carbonate, saturated. NaCl, 0.1 M sodium phosphate and saturated. NaCl aqueous solutions and dried (Na₂SO₄). Removal of the solvent and purification by chromatography on silica gel (0-4% methanol gradient over CH₂Cl₂) yielded 2-(10H-indolo[3,2-b]quinoline-11-carboxamide)acetic methyl ester (0.1, 18%). as a foam. Both fractions were combined yielding 0.4 g (75% yield). HPLC (conditions: HPLC solutions were solvent A: 5% acetonitrile in 100 mM triethylammonium acetate, pH 6.5 and solvent B: 70% acetonitrile in 100 mM triethylammonium acetate (pH 6.5). Column: PRP-1(Hamilton) 250×10 mm. Flow rate 3 ml/min linear gradient from 15 to 80 in B) single peak of retention time 18.8 min. ¹H-NMR (CD₃OD, δ, ppm): 8.45 (d, J=8.0 Hz, 1H), 8.40 (d, J=8.0 Hz, 1H), 8.16 (d, J=9.2 Hz, 1H), 7.8-7.6 (m, 4H), 7.3 (dd, J=8.0 Hz, 1H), 4.38 (s, 2H), 3.89 (s, 3H).

2-(10H-indolo[3,2-b]quinoline-11-carboxamide)acetic methyl ester (0.38, 1.14 mmol) were dissolved in 9.5 ml of dioxane and a 1M solution of lithium hydroxide (0.45 g in 19 ml) of a 1:1 water/methanol solution was added slowly. After 1.5 h of magnetic stirring, the solution was neutralized with a 2 M NaH₂PO₄ solution until pH 5.0 and some drops of diluted HCl to reach pH 3.0. A small precipitate formed, the solution was filtered and the precipitate was discarded. To the resulting aqueous solution ethyl acetate was added. The organic layer was separated and the aqueous phase was washed twice with ethyl acetate. The resulting organic phases are combined and dried (Na₂SO₄) yielding 2-(10H-indolo[3,2-b]quinoline-11carboxamide)acetic acid (0.36 g, 99%) as a reddish solid. HPLC (conditions described above) single peak of retention time 10.5 min. ¹H-NMR (CD₃OD, δ, ppm): 11.8 (wide s, 1H), 8.9 (m, 1H, NH), 8.2 (m, 2H), 8.07 (d, 1H), 7.7-7.5 (m, 4H), 7.2 (m, 1H), 3.92 (d, 2H). MS (Cl/NH₃) found 320.0, expected for C₁₈H₁₃N₃O₃ 319.

### Example 2

### Preparation of 2-(acridine-9-carboxamide)acetic acid (Compound 1d)

Acridine-9-carboxylic acid (Compound 1b, 0.5 g, 2.24 mmol) was dissolved in 20 ml of dimethylformamide together with N,N-diisopropylcarbodiimide (0.35 ml, 2.24 mmol) and 1-hydroxybenzotriazole (0.30 g, 2.24 mmol). The mixture was stirred for 10 minutes. To this solution a mixture of glycine methyl ester hydrochloride (0.2 g, 2.24 mmol) and N,N-diisopropylethylamine (0.39 ml, 2.24 mmol) dissolved in dimethylformamide was added. After 2 hrs of magnetic stirring at room temperature N,N-diisopropylethylamine (0.2 ml, 1.12 mmol) were added and stirring was continued for 1 hour. The resulting mixture was concentrated to dryness and the residue was dissolved in ethyl acetate. The organic phase was washed with 5% sodium carbonate, saturated. NaCl, 0.1M sodium phosphate and saturated. NaCl aqueous solutions and dried (Na₂SO₄). Removal of the solvent and purification by chromatography on silica gel (0-4% methanol gradient over CH₂Cl₂) yielded 2-(acridine-9-carboxamide)acetic methyl ester (0.69, 70%) as a foam. HPLC (conditions in example 1) single peak of retention time 10.5 min. ¹H-NMR [CDCl₃, δ, ppm]: 8.07 (d, 2H), 8.0 (d, 2H), 7.64 (m, 2H), 7.46 (m, 2H), 4.35(s, 2H), 3.78 (s, 3H). ¹³C-NMR [CDCl₃, δ, ppm]: 168, 166, 148.0, 130.7, 128.7, 126.9, 125.3, 122.3, 52.5, 41.7.

2-(acridine-9-carboxamide)acetic methyl ester (0.44, 1.49 mmol) were dissolved in 11 ml of dioxane and cooled with ice. 1M solution of lithium hydroxide (0.52 g in 22 ml) of a 1:1 water/methanol solution was added to the solution slowly. After 30 min of magnetic stirring, the solution was neutralized with a 2 M NaH₂PO4 solution until pH 5.0. A small precipitate formed, the solution was filtered and the precipitate was discarded. To the resulting aqueous solution diluted HCl was added until reaching pH 3.0 and a big precipitate is formed yielding 2-(acridine-9-carboxamide)acetic acid (0.23 g, 55% as a red solid. UV (λ max): 240, 359 nm. HPLC (conditions in example 1) single peak of retention time 7.6 min. ¹H-NMR [DMSO-d₆, δ, ppm]: 9.46 (m, 1H), 8.27 (d, 2H), 8.21 (d, 2H), 7.9 (m, 2H), 7.6 (m, 2H), 4.17 (m, 2H). ¹³C-NMR [DMSO-d₆, δ, ppm]: 171.2, 166.8, 148.2, 130.8, 129.2, 126.7, 126.1, 122.0, 41.6. MS (Cl/NH₃) found 281.0, expected for C₁₆H₁₂N₂O₃ 280.

### Example 3

### N-(1,3-dihydroxybutan-2-yl)-10H-indolo[3,2-d]quinoline-11-carboxamide (Compound 2a, monomer Qut)

10H-indolo[3,2-d]quinoline-11-carboxylic acid (Compound 1a, 0.25 g, 0.95 mmol) was dissolved in 10 ml of dimethylformamide together with N,N-diisopropylcarbodiimide (0.15 ml, 0.95 mmol) and 1-hydroxybenzotriazole (HOBt) (0.128 g, 0.95 mmol). The mixture was stirred for 10 minutes and L-threoninol (50 mg, 0.47 mmol) was added. After 24 hrs of magnetic stirring at room temperature the mixture was concentrated to dryness. The product was crystallized from chloroform yielding N-(1,3-dihydroxybutan-2-yl)-10H-indolo[3,2-d]quinoline-11-carboxamide (300 mg, 90%) of a red solid. HPLC (conditions in example 1) single peak of retention time 14.9 min. ¹H-NMR [DMSO-d₆, δ, ppm]: 8.41 (wide d, 1H, NH), 8.2 (m, 2H), 7.9 (m, 1H), 7.3-7.6 (m, 5H), 5.44 (wide, 2H, OH), 4.14 (m, 1H, CH), 3.99 (m, 1H, CH), 3.4-3.6 (m, 2H, CH₂), 1.21 (d, J=6.6 Hz, 3H, CH₃). MS (Cl/NH₃) found 350.1, expected for C₂₀H₁₉N₃O₃ 349.

### Example 4

### N-(1,3-dihydroxybutan-2-yl)acridine-9-carboxamide (Compound 2b, monomer Act).

Acridine-9-carboxylic acid (Compound 1b, 0.5 g, 2.24 mmol) was dissolved in 20 ml of dimethylformamide together with N,N-diisopropylcarbodiimide (0.35 ml, 2.24 mmol) and 1-hydroxybenzotriazole (0.303 g, 2.24 mmol). The mixture was stirred for 10 minutes and L-threoninol (117 mg, 1.12 mmol) was added. After 24 hrs of magnetic stirring at room temperature the mixture was concentrated to dryness. Removal of the solvent and purification by chromatography on silica gel (0-2% methanol gradient over CH₂Cl₂) yielded N-(1,3-dihydroxybutan-2-yl)acridine-9-carboxamide (0.69 g, 70%) as a foam. HPLC (conditions in example 1) single peak of retention time 9.1 min. ¹H-NMR [CDCl₃, δ, ppm]: 8.82 (m, 1H, NH), 8.35 (m, 2H), 8.05 (m, 2H), 7.8 (m, 2H), 7.6 (m, 2H), 5.3 (s, 2H, OH), 4.21 (m, 1H, CH), 3.85 (m, 1H, CH), 3.49 (m, 2H, CH₂), 1.26 (d, 3H, CH₃). MS (Cl/NH₃) found 311.1, expected for C₁₈H₂₀N₂O₃ 312.

### Example 5

### N-((1,3-dihydroxybutan-2-ylcarbamoyl)methyl)-10H-indolo[3,2-d]quinoline-11-carboxamide (2c, monomer Qqt).

2-(10H-Indolo[3,2-b]quinoline-11carboxamide)acetic acid (Compound 1c, 0.32 g, 1 mmol) was dissolved in 10 ml of dimethylformamide together with N,N-diisopropylcarbodiimide (0.15 ml, 1 mmol) and 1-hydroxybenzotriazole (0.13 g, 1 mmol). The mixture was stirred for 10 minutes and L-threoninol (105 mg, 1 mmol) was added. After 24 hrs of magnetic stirring at room temperature the mixture was concentrated to dryness. The product was crystallized from CH₂Cl₂ yielding N-((1,3-dihydroxybutan-2-ylcarbamoyl)methyl)-10H-indolo[3,2-d]quinoline-11-carboxamide (380 mg, 93%) of a red solid. HPLC (conditions in example 1) single peak of retention time 10.9 min.¹H-NMR [DMSO-d6, δ-ppm]: 11.5 (s, 1H), 9.2 (t, 1H), 8.5 (d, 1H), 8.2 (d, 1H), 7.7 (m, 4H), 7.3 (m,2H), 4.2 (d, 1H), 4.0 (m, 1H), 3.8 (m, 1H), 3.7 (d, 2H), 3.6 (d, 1H), 1.1 (d, 3H). MS (Cl/NH₃) found 395.2, expected for C₂₂H₂₂N₄O₅ 394.

### Example 6

### N-((1,3-dihydroxybutan-2-ylcarbamoyl)methyl)acridine-9-carboxamide (2d, monomer Aqt).

2-(Acridine-9-carboxamide)acetic acid (Compound 1d, 0.2 g, 0.67 mmol) was dissolved in 30 ml of dimethylformamide together with N,N-diisopropylcarbodiimide (0.1 ml, 0.67 mmol) and 1-hydroxybenzotriazole (0.09 g, 0.67 mmol). The mixture was stirred for 10 minutes and L-threoninol (105 mg, 1 mmol) was added. After 24 hrs of magnetic stirring at room temperature the mixture was concentrated to dryness. The product was crystallized from CH₂Cl₂ yielding N-((1,3-dihydroxybutan-2-ylcarbamoyl)methyl)acridine-9-carboxamide (250 mg, 97%) of a red solid. HPLC (conditions in example 1) single peak of retention time 9.3 min. ¹H-NMR [DMSO-d₆, δ, ppm]: 8.28 (d, 2H), 8.10 (d, 2H), 7.8 (m, 2H), 7.58 (m, 2H), 4.5 (wide, 1H, OH), 4.24 (m, 1H), 4.0 (m, 1H), 3.8 (m, 1H), 3.6-3.4 (m, CH₂), 1.14 (d, 2H, CH₃). MS (Cl/NH₃) found 368.1, expected for C₂₀H₂₁N₃O₅ 367.

### Example 7

### N-(3-hydroxy-1-(4,4′-dimethoxytrityl)oxybutan-2-yl)-10H-indolo[3,2-d]quinoline-11-carboxamide (Compound 3a)

N-(1,3-dihydroxybutan-2-yl)-10H-indolo[3,2-d]quinoline-11-carboxamide (Compound 2a, 0.31 g, 1 mmol) was reacted with 4,4-dimethoxytrityl chloride (0.33 g, 1 mmol) and N,N-dimethylaminopiridine (6 mg, 0.005 mmol) in 20 ml of pyridine. After 2 hr of magnetic stirring at room temperature, 84 mg (0.25 mmol) of 4,4-dimethoxytrityl chloride were added and the mixture was stirred for 30 min. The reaction was stopped with the addition of 0.5 ml of methanol and the mixture was concentrated to dryness. The residue was dissolved in CH₂Cl₂ and washed with 5% sodium bicarbonate and brine and dried. The resulting product was purified by chromatography on silica gel (0-2% methanol in CH₂Cl₂ with a 1% triethylamine) yielding 110 mg of N-(3-hydroxy-1-(4,4′-dimethoxytrityl)oxybutan-2-yl)-10H-indolo[3,2-d]quinoline-11-carboxamide (18%) as a foam. ¹H-NMR [CDCl₃, δ-ppm]: 9.3 (s, 1H), 8.5 (d, 1H), 8.3 (d, 1H), 8.4 (d, 1H), 7.7-7.2 (14H, aromatics), 6.8 (m, 4H), 4.4 (d, 1H), 4.2 (m, 1H), 3.8 (m,1H), 3.7 (s, 6H), 3.3 (s, 1H), 1.1 (d, 3H).

### Example 8

### N-(3-(N,N-diisopropylamino-2-cyanoethoxyphosphinyl)oxy-1-(4,4′-dimethoxytrityl)oxybutan-2-yl)-10H-indolo[3,2-d]quinoline-11-carboxamide (Compound 4a).

N-(3-hydroxy-1-(4,4′-dimethoxytrityl)butan-2-yl)-10H-indolo[3,2-d]quinoline-11-carboxamide (Compound 3a, 220 mg, 0.38 mmol) was dried by coevaporation of dry acetonitrile and dissolved in 10 ml of dry dichloromethane. N,N-Diisopropylethylamine (0.2 ml, 1.14 mmol) was added and the mixture was purged with argon and cooled with an ice-water bath. O-2-Cyanoethyl-N,N-diisopropyl-chlorophosphoramidite (0.13 ml, 0.57 mmol) was added dropwise with continuous stirring. After the addition of the chlorophosphine, the mixture was allowed to warm to room temperature and stirred for 2 hr. The reaction was stopped by addition of 20 ml of CH₂Cl₂ containing 1% of triethylamine and the mixture washed with brine and dried. The resulting product was purified by chromatography on silica gel (hexane/ethyl acetate (2:1)+1% triethylamine) yielding 160 mg of N-(3-(N,N-diisopropylamino-2-cyanoethoxyphosphinyl)oxy-1-(4,4′-dimethoxytrityl)oxybutan-2-yl)-10H-indolo[3,2-d]quinoline-11-carboxamide (53%) as a foam. ³¹P-NMR [CDCl₃, δ, ppm]: 148.4. ¹H-NMR [CDCl₃, δ-ppm]: 9.3 (s, 1H), 8.5 (d, 1H), 8.3 (d, 1H), 8.4 (d, 1H), 7.7-7.2 (14H, aromatics), 6.8 (m, 4H), 4.7 (m, 2H), 4.4 (d, 1H), 4.2 (m, 1H), 3.8 (m, 1H), 3.7 (s, 6H), 3.5 (m, 2H), 2.12 (t, 2H), (s, 1H), 1.1 (d, 3H).

### Example 9

### N-(3-hydroxy-1-(4,4′-dimethoxytrityl)oxybutan-2-yl)acridine-9-carboxamide (Compound 3b)

N-(1,3-dihydroxybutan-2-yl)acridine-9-carboxamide (Compound 2b, 0.45 g, 1.45 mmol) was reacted with 4,4-dimethoxytrityl chloride (0.54 g, 1.6 mmol) and N,N-dimethylaminopiridine (10 mg, 0.084 mmol) in 20 ml of pyridine. After 4 hr of magnetic stirring at room temperature, the reaction was stopped with the addition of 1 ml of methanol and the mixture was concentrated to dryness. The residue was dissolved in CH₂Cl₂ and washed with 5% sodium bicarbonate and brine and dried. The resulting product was purified by chromatography on silica gel (0-2% methanol in CH₂Cl₂ with a 1% triethylamine) yielding 490 mg of N-(3-hydroxy-1-(4,4′-dimethoxytrityl)oxybutan-2-yl)acridine-9-carboxamide (55%) as a foam. ¹H-NMR [CDCl₃, δ, ppm]: 8.3-8.2 (m, 2H), 8.1 (m, 2H), 7.8 (m, 2H), 7.5-7.1 (m, 11H), 6.8-6.7 (m, 4H), 4.5 (m, 1H, OH), 4.2 (m, 1H), 3.7 (m, 7H), 3.5 (m, 2H, CH₂), 1.1 (d, 3H, CH₃).

### Example 10

### N-(3-(N,N-diisopropylamino-2-cyanoethoxyphosphinyl)oxy-1-(4,4′-dimethoxytrityl)oxybutan-2-yl)acridine-9-carboxamide (Compound 4b)

N-(3-hydroxy-1-(4,4′-dimethoxytrityl)oxybutan-2-yl)acridine-9-carboxamide (Compound 3b, 190 mg, 0.31 mmol) was dried by coevaporation of dry acetonitrile and dissolved in 5 ml of dry dichloromethane. N,N-Diisopropylethylamine (0.33 ml, 1.8 mmol) was added and the mixture was purged with argon and cooled with an ice-water bath. O-2-Cyanoethyl-N,N-diisopropyl-chlorophosphoramidite (0.20 ml, 0.9 mmol) was added dropwise with continuous stirring. After the addition of the chlorophosphine, the mixture was allowed to warm to room temperature and stirred for 1.5 hr. The reaction was stopped by addition of 20 ml of CH₂Cl₂ containing 1% of triethylamine and the mixture washed with brine and dried. The resulting product was purified by chromatography on silica gel (hexane/ethyl acetate (3:2)+1% triethylamine) yielding 210 mg of N-(3-(N,N-diisopropylamino-2-cyanoethoxyphosphinyl)oxy-1-(4,4′-dimethoxytrityl)oxybutan-2-yl)acridine-9-carboxamide (82%) as a foam. ³¹P-NMR [CDCl₃, δ, ppm]: 149.1, 148.3 (two diastereoisomers). ¹H-NMR [CDCl₃, δ, ppm]: 8.3-8.2 (m, 2H), 8.1 (m, 2H), 7.8 (m, 2H), 7.5-7.1 (m, 11H), 6.8-6.7 (m, 4H), 4.7 (m, 2H), 4.2 (m, 1H), 3.7 (m, 7H), 3.5 (m, 4H), 2.2 (t, 2H), 1.1-1.0 (m, 15H).

### Example 11

### N-(3-hydroxy-1-(4,4′-dimethoxytrityl)oxybutan-2-ylcarbamoyl)methyl)-10H-indolo[3,2-d]quinoline-11-carboxamide (Compound 3c).

N-((1,3-dihydroxybutan-2-ylcarbamoyl)methyl)-10H-indolo[3,2-d]quinoline-11-carboxamide (Compound 2c, 170 mg, 0.34 mmol) was reacted with 4,4-dimethoxytrityl chloride (0.17 g, 0.51 mmol) and N,N-dimethylaminopiridine (3.5 mg) in 10 ml of pyridine. After 5 hr of magnetic stirring at room temperature, the reaction was stopped with the addition of 1 ml of methanol and the mixture was concentrated to dryness. The residue was dissolved in CH₂Cl₂ and washed with 5% sodium bicarbonate and brine and dried. The resulting product was purified by chromatography on silica gel (0-5% methanol in CH₂Cl₂) yielding 120 mg of N-(3-hydroxy-1-(4,4′-dimethoxytrityl)oxybutan-2-ylcarbamoyl)methyl)-10H-indolo[3,2-d]quinoline-11-carboxamide (56%) as a foam. ¹H-NMR [CDCl₃, δ, ppm]: 11.7 (s, 1H, NH), 9.3 (m, 1H, NH), 8.6 (d, 2H), 8.4 (d, 2H), 8.1 (d, 1H), 7.9-7.0 (m, 16H), 5.7 (wide, 1H, OH), 4.41 (m, 2H, CH₂), 4.2 (m, 1H, CH), 3.9 (m, 1H, CH), 3.7-3.5 (m, 8H, MeO DMT+CH₂) 1.2 (d, CH₃).

### Example 12

### N-(3-hydroxy-1-(4,4′-dimethoxytrityl)oxybutan-2-ylcarbamoyl)methyl)acridine-9-carboxamide (Compound 3d)

N-((1,3-dihydroxybutan-2-ylcarbamoyl)methyl)acridine-9-carboxamide (Compound 2d, 0.14 g, 0.38 mmol) was reacted with 4,4-dimethoxytrityl chloride (0.13 g, 0.39 mmol) and N,N-dimethylaminopiridine (2.3 mg, 0.019 mmol) in 10 ml of pyridine. After 3 hr of magnetic stirring at room temperature, 10 mg (0.03 mmol) of 4,4-dimethoxytrityl chloride were added and the mixture was stirred for 30 min. The reaction was stopped with the addition of 1 ml of methanol and the mixture was concentrated to dryness. The residue was dissolved in CH₂Cl₂ and washed with 5% sodium bicarbonate and brine and dried. The resulting product was purified by chromatography on silica gel (0-2% methanol in CH₂Cl₂ with a 1% triethylamine) yielding 140 mg of N-(3-hydroxy-1-(4,4′-dimethoxytrityl)oxy-butan-2-ylcarbamoyl)methyl)acridine-9-carboxamide (50%) as a foam. ¹H-NMR [CDCl₃, δ, ppm]: 8.22 (d, 2H), 8.12 (d, 2H), 7.78 (m, 2H), 7.5 (m, 2H), 7.4-7.1 (m, 9H, DMT), 6.82 (m, 4H, DMT), 6.6 (m, 1H, OH), 4.38 (m, 2H, CH₂), 4.18 (m, 1H, CH), 4.0 (m, 1H, CH), 3.74 (s, 6H, MeO DMT), 3.4 (m, 2H, CH₂), 1.18 (d, 3H, CH₃).

### Example 13

### N-((2S, 3R)-1,3-dihydroxybutan-2-yl)-2-phenylquinoline-4-carboxamide (Compound 2e, monomer Pht)

2-Phenyl-4-quinolinecarboxylic acid (compound 1e, 1.1 g, 4.7 mmol) was dissolved in DMF (10 mL). To this solution, 0.6 mL (4.7 mmol) of N,N′-diisopropylcarbodiimide and 0.63 g (4.7 mmol) of HOBt were added followed by 0.5 g (4.7 mmol) of (L)-threoninol. The resulting mixture was stirred overnight at room temperature and then concentrated to dryness. The residue was dissolved in CH₂Cl₂ and the product was precipitated by addition of saturated solution of NaHCO₃. The resulting precipitated was filtered and washed with Et₂O to yield the desired compound (1 g, 63%) as a white solid. HPLC (conditions in example 1) single peak of retention time 12.2 min. ¹H NMR [MeOD δ, ppm]: 8.27-8.12 (m, 4H), 7.83 (m, 1H), 7.81-7.52 (m, 5H), 5.32 (m, 1H, (CHNH), 4.24 (m, 1H, (CHOH), 4.23-4.12 (m, 2H, (CH₂OH), 1.35 (d, J=6.5 Hz, 3H, CH₃). MS (Cl/NH₃) found 337.1, expected for C₂₀H₂₀N₂O₃ 336.3.

### Example 14

### N-((2S, 3R)-1-(4,4′-dimethoxytrityloxy)-3-hydroxybutan-2-yl)-2-phenylquinoline-4-carboxamide (Compound 3e)

N-((2S, 3R)-1,3-dihydroxybutan-2-yl)-2-phenylquinoline-4-carboxamide (compound 2g, 0.5 g, 1.48 mmol) was reacted with 4,4-dimethoxytrityl chloride (0.6 g, 1.78 mmol) and N,N-dimethylaminopiridine (10.3 mg, 0.08 mmol) in 20 ml of pyridine. After 4 hr of magnetic stirring at room temperature, 100 mg (0.3 mmol) of 4,4-dimethoxytrityl chloride were added and the mixture was stirred for 1 hr. The reaction was stopped with the addition of 1 ml of methanol and the mixture was concentrated to dryness. The residue was dissolved in CH₂Cl₂ and washed with 5% sodium bicarbonate and brine and dried. The resulting product was purified by chromatography on silica gel (0-2% methanol in CH₂Cl₂) yielding 0.9 g of N-((2S, 3R)-1-(4,4′-dimethoxytrityloxy)-3-hydroxybutan-2-yl)-2-phenylquinoline-4-carboxamide (95%) as a foam. ¹H-NMR [CDCl₃, δ, ppm]: 8.26 (d, 1H, Ar), 8.18 (d, 1H, Ar) 8.08 (m, 2H, Ar) 7.9 (s, 1H, Ar), 7.8-7.1 (m, 14H, Ar, DMT), 6.8 (m, 4H, DMT), 4.6 (m, 1H, OH), 4.25 (m, 1H, CH), 3.8-3.6 (m, 7H, MeO DMT, CH), 3.4 (m, 2H, CH₂), 1.15 (d, 3H, CH₃).

### Example 15

### N-((2S, 3R)-3-(N,N-diisopropylamino-2-cyanoethoxyphosphinyl)oxy-1-(4,4′-dimethoxytrityloxy)-butan-2-yl)-2-phenylquinoline-4-carboxamide (compound 4e)

N-((2S, 3R)-1-(4,4′-dimethoxytrityloxy)-3-hydroxybutan-2-yl)-2-phenylquinoline-4-carboxamide (280 mg, 0.44 mmol) was dried by coevaporation of dry acetonitrile and dissolved in 5 ml of dry dichloromethane. N,N-Diisopropylethylamine (0.23 ml, 1.3 mmol) was added and the mixture was purged with argon and cooled with an ice-water bath. 0-2-Cyanoethyl-N,N-diisopropyl-chlorophosphoramidite (0.15 ml, 0.65 mmol) was added dropwise with continuous stirring. After the addition of the chlorophosphine, the mixture was allowed to warm to room temperature and stirred for 30 min. The reaction was stopped by addition of 20 ml of CH₂Cl₂ containing 1% of triethylamine and the mixture washed with brine and dried. The resulting product was purified by chromatography on silica gel (hexane/ethyl acetate (3:1)+1% triethylamine) yielding 220 mg of N-((2S, 3R)-3-(N,N-diisopropylamino-2-cyanoethoxyphosphinyl)oxy-1-(4,4′-dimethoxytrityloxy)-butan-2-yl)-2-phenylquinoline-4-carboxamide (60%) as a foam. ³¹P-NMR [CDCl₃, δ, ppm]: 148.5. ¹H-NMR [CDCl₃, δ, ppm]: 8.24 (m, 1H, Ar), 8.16 (m, 1H, Ar), 7.9 (s, 1H, Ar), 7.8 (m, 1H, Ar), 7.6-7.1 (m, 15H, Ar, DMT), 6.8 (m, 4H, DMT), 4.3 (m, 1H, CH), 4.1 (m, 2H, CH₂), 3.8-3.4 (m, 11H, MeO DMT, CH isopropyl, CH₂), 2.1 (m, 2H, CH₂CN), 1.3-1.1 (m, 15H, CH₃ threoninol and CH₃ isopropyl).

### Example 16

### N-((2S, 3R)-1,3-dihydroxybutan-2-yl)-(4a, 10a-dihydro-10,11-diazabenzo[b]fluoren-10-yl)acetamide (Compound 2f, monomer Nct)

(4a, 10a-Dihydro-10,11-diazabenzo[b]fluoren-10-yl) acetic acid (compound 1f, 0.27 g, 1.0 mmol) was dissolved in DMF (10 mL). To this solution, 0.15 ml (1 mmol) of N,N′-diisopropylcarbodiimide and 0.135 g (1 mmol) of HOBt were added followed by 0.10 g (1 mmol) of (L)-threoninol. The resulting mixture was stirred overnight at room temperature and then concentrated to dryness. The residue was treated with CH₂Cl₂, and the desired product was not soluble. The resulting solution containing the impurities was filtered out and the residual solid was isolated yielding 0.19 g (52% yield) of a brown solid that was used in the following step without further purification. TLC (5% methanol in CH₂Cl₂) Rf 0.31. HPLC (conditions in example 1) single peak of retention time 15.1 min ¹H NMR [CD₃OD δ, ppm]: 9.0-7.3 (m, 9H), 5.7 (s, 2H), 4.0 (m, 1H), 3.8 (m, 1H), 2.9 (dd, 2H, CH₂OH), 1.10 (d, 3H, CH₃). MS (Cl/NH₃) found 364.3, expected for C₂₁H₂₁ N₃O₃ 363.2.

### Example 17

### N-((2S, 3R)-1-bis(4-methoxyphenyl)-phenyl-methoxy)-3-hydroxybutan-2-yl)-2-(4a, 10a-dihydro-10,11-diazabenzo[b]fluoren-10-yl) acetamide (Compound 3f)

N-((2S, 3R)-1,3-dihydroxybutan-2-yl)-(Neocriptolepinyl)acetamide (compound 2f, 0.19 g, 0.52 mmol) was reacted with 4,4-dimethoxytrityl chloride (0.21 g, 0.62 mmol) and N,N-dimethylaminopiridine (3.6 mg) in 20 ml of pyridine. After 5 hr of magnetic stirring at room temperature, the reaction was stopped with the addition of 1 ml of methanol and the mixture was concentrated to dryness. The residue was dissolved in CH₂Cl₂ and washed with brine and dried. The resulting product was purified by chromatography on silica gel (0-1% methanol in CH₂Cl₂) yielding 30 mg of N-((2S, 3R)-1-bis(4-methoxyphenyl)-phenyl-methoxy)-3-hydroxybutan-2-yl)-2-(4a, 10a-dihydro-10,11-diazabenzo[b]fluoren-10-yl) acetamide (9%). ¹H NMR [CDCl₃ δ, ppm]: 8.7-6.9 (m, 18H), 6.45 (d, 4H), 5.1 (dd, 2H), 3.9 (m, 1H), 3.8 (m, 1H), 3.7 (s, 6H, OMe DMT), 3.0 (m, 2H, CH₂OH), 1.10 (d, 3H, CH₃).

### Example 18

### (R)-3-(Acridin-9-ylamino)propane-1,2-diol (Compound 5a)

9-chloro-acridine (1 g, 4.68 mmol) and (R)-3-amino-propane-1,2-diol (0.42 g, 4.68 mmol) in 2-ethoxyethanol (40 ml) was heated at 120° C. for 30 min. Removal of the solvent yielded the desired compound (0.9 g, 75%) as a yellow solid. HPLC (conditions in example 1) single peak of retention time 8.4 min. ¹H-NMR [MeOD δ, ppm]: 8.01 (m, 2H), 7.84 (m, 2H), 7.60 (m, 2H), 4.37-4.28 (m, 2H, (CH₂NH), 4.17 (m, 1H, CHOH), 3.82-3.75 (m, 2H, CH₂OH). MS (Cl/NH₃) found 269.1, expected for C₁₆H₁₆N₂O₂ 268.3.

### Example 19

### (R)-3-(Acridin-9-ylamino)-1-(4,4′-dimethoxytrityl)oxypropane-2-ol (Compound 6a)

(R)-3-(acridin-9-ylamino)propane-1,2-diol (compound 5a, 400 mg, 1.49 mmols) was dried by coevaporation of dry pyridine and dissolved in 20 ml of dry pyridine. N,N-dimethyaminopyridine (10.4 mg, 0.085 mmols) was added and then 4,4′-dimetoxytrityl chloride (555 mg, 1.63 mmols). After 5 h of magnetic stirring at room temp. the reaction was stopped with the addition of 1 ml of methanol and the mixture was concentrated to dryness. The residue was dissolved in dichloromethane and washed with 5% sodium bicarbonate and brine and dried. The resulting product was purified by chromatography on silica gel (0-1.5% methanol in dichloromethane with 1% triethylamine) yielding 320 mg of (R)-3-(Acridin-9-ylamino)-1-(4,4′-dimethoxytrityl)oxypropane-2-ol (37.6%) as a foam. ¹H-NMR [CDCl₃, δ-ppm]: 8.1 (m, 2H), 7.8 (m, 2H), 7.1-7.4 (m, 13H arom), 6.7 (m, 4H, DMT), 4.2 (m, 2H, CH₂CN), 3.7 (s, 6H, OMe), 3.4-3.6 (m, 4H, CH₂O, CH₂ODMT)

### Example 20

### O-[(R)-3-(Acridin-9-ylamino)-1-(4,4′-dimethoxytrityl)oxypropane-2-yl]-N,N-diisopropylamino-2-cyanoethoxyphosphoramidite (Compound 7a)

(R)-3-(Acridin-9-ylamino)-1-(4,4′-dimethoxytrityl)oxypropane-2-ol (compound 6a, 250 mg, 0.44 mmols) was dried by coevaporation of dry acetonitrile and dissolved in 5 ml of dry dichloromethane. N,N-Diisopropylethylamine (0.228 ml, 1.57 mmol) was added and the mixture was purged with argon and cooled with an ice-water bath. O-2-Cyanoethyl-N,N-diisopropyl-chlorophosphoramidite (0.146 ml, 0.65 mmol) was added dropwise with continuous stirring. After the addition of the chlorophosphine, the mixture was allowed to warm to room temperature and stirred for 1 hr. The reaction was stopped by addition of 50 ml of CH₂Cl₂ containing 1% of triethylamine and the mixture washed with brine and dried. The resulting product was purified by chromatography on silica gel (0-1.5% methanol in dichloromethane with 1 triethylamine) yielding 300 mg of O-[(R)-3-(Acridin-9-ylamino)-1-(4,4′-dimethoxytrityl)oxypropane-2-yl]-N,N-diisopropylamino-2-cyanoethoxyphosphoramidite (90%) as a foam. ¹H-NMR [CDCl₃, δ-ppm]: 8.1 (m, 2H), 7.8 (m, 2H), 7.2-7.4 (m, 13H arom), 6.7 (m, 4H, DMT), 4.2 (m, 3H, CH, CH₂), 3.7-3.4 (m, 12H, OMe DMT, 2 CH isopropyl, 2 CH₂), 2.8 (m, 2H), 1.25 (m, 12H). ³¹P-NMR [CDCl₃, δ, ppm]: 150.0, 149.3 (two diastereoisomers).

### Example 21

### (S)-3-(5-Methyl-5H-indolo[3,2b]quinolin-11-ylamino)propane-1,2-diol (Compound 5b)

A solution of 11-chloro-5-metil-5H-indolo[3,2-b]quinoline (20 mg, 0.075 mmol) and (S)-3-amino-propane-1,2-diol (29 mg, 0.32 mmol) in 2-ethoxyethanol (10 ml) was heated at 120° C. for 10 min. Removal of the solvent yielded the desired compound (15 mg, 63%) as an orange solid. ¹H NMR [CDCl₃ δ, ppm]: 8.58 (m, 2H), 8.31-8.03 (m, 2H), 7.77 (m, 3H), 7.43 (m, 1H), 4.77 (s, 3H, NCH₃), 4.25 (m, 2H, CH₂), 3.69 (m, 1H, CH), 3.58-3.50 (m, 2H, CH₂). MS (Cl/NH₃) found 322.2, expected for C₁₉H₁₉N₃O₂321.3.

### Example 22

### (S)-3-(5-Methyl-5H-indolo[3,2b]quinolin-11-ylamino)-1-(4,4′-dimethoxytrityl)oxypropane-2-ol (Compound 6b)

(S)-3-(5-Methyl-5H-indolo[3,2b]quinolin-11-ylamino)propane-1,2-diol (compound 5b, 300 mg, 0.93 mmol) was reacted with dimethoxytrityl chloride using N,N,-dimethylaminopyridine as catalyst in pyridine as described in example 7. Yield: 28%. ¹H-NMR [Cl₃CD δ, ppm]: 13.1 (s, 1H, NH), 9.2 (m, 1H), 8.6 (m, 1H), 8.2 (m, 1H), 7.9 (m, 2H), 7.7 (m, 1H), 7.5 (m, 2H), 7.3-7.1 (m, 9H, DMT), 6.8-6.7 (m, 4H, DMT), 4.3-4.1 (m, 1H, CH), 3.7 (s, 6H, MeO DMT), 3.5-3.3 (m, 2H, CH₂).

### Example 23

### [{2-[Acridine-9-carboxamide]-acetyl}-(2-tert-butoxycarbonylamino-ethyl)-amino]-acetic acid methyl ester (Compound 8a)

2-(Acridine-9-carboxamide)acetic acid (1d) (0.094 g, 0.33 mmol) was dissolved in DMF (2 mL). To this solution, 0.084 ml (0.5 mmol) of N-ethylmorpholine and 0.055 g (0.33 mmol) of HOOBt were added followed by a solution of 0.064 g (0.27 mmol) of methyl N-[2-(tert-butoxycarbonyl-amino)ethyl]glycinate (c.f. P. Clivio et al. “Synthesis and photochemical behaviour of peptide nucleic acid dimmers and analogues containing 4-thiothymine: Unprecedent (5-4) photoadduct reversion” J. Am. Chem. Soc. 1998, vol. 120, 1157-1166) in 2 ml of DMF and 0.053 ml (0.42 mmol) of N,N′-diisopropylcarbodiimide. The resulting mixture was stirred overnight at room temperature and then concentrated to dryness. The residue was dissolved in CH₂Cl₂ and the solution was washed with water and brine. The organic phase was dried and concentrated to dryness. The resulting product was purified on alumina (CH₂Cl₂:CH₃OH/99:1) yielded the desired compound (64 mg, 40%) as a foam. ¹H-NMR [MeOD δ, ppm]: 8.41 (m, 2H), 8.21 (m, 2H), 7.91 (m, 2H), 7.67 (m, 2H), 4.61 (s, 2H, (CH₂CON), 4.43 (s, 2H, (CH₂COOCH₃), 3.82 (s, 3H, COOCH₃), 3.65 and 3.33 (m, 4H, NCH₂CH₂NH), 1.49 (bs, 9H, C(CH₃)₃). MS (Cl/NH₃) found 495.2, expected for C₂₆H₃₀N₄O₆ 494.5.

### Example 24

### [(2-tert-Butoxycarbonylamino-ethyl)-{2-[10H-indolo[3,2-b]quinoline-11-carbonyl)amino]-acetyl}-amino)acetic acid methyl ester (Compound 8b)

2-(10H-indolo[3,2-b]quinoline-11carboxamide)acetic acid (1c) (0.061 g, 0.19 mmol) was dissolved in DMF (2 ml). To this solution, 0.049 ml (0.43 mmol) of N-ethylmorpholine and 0.032 g (0.19 mmol) of HOOBt were added followed by a solution of 0.045 g (0.19 mmol) of methyl N-[2-(tert-butoxycarbonyl-amino)ethyl]glycinate (c.f. P. Clivio et al. “Synthesis and photochemical behaviour of peptide nucleic acid dimmers and analogues containing 4-thiothymine: Unprecedent (5-4) photoadduct reversion” J. Am. Chem. Soc. 1998, vol. 120, 1157-1166) in 2 ml of DMF and 0.031 ml (0.24 mmol) of N,N′-diisopropylcarbodiimide. The resulting mixture was stirred overnight at room temperature and then concentrated to dryness. The residue was dissolved in CH₂Cl₂ and the solution was washed with water and brine. The organic phase was dried and concentrated to dryness. The resulting product was purified on alumina (CH₂Cl₂:CH₃OH/99:1) yielded the desired compound (62 mg, 62%) as a foam. ¹H-NMR [MeOD δ, ppm]: 8.51 (m, 1H), 8.42 (m, 1H), 8.29 (m, 1H), 7.75 (m, 1H), 7.68 (m, 2H), 7.60 (m, 1H), 7.36 (m, 1H), 4.63 (s, 2H, (CH₂CON), 4.45 (s, 2H, (CH₂COOCH₃), 3.82 (s, 3H, COOCH₃), 3.66 and 3.33 (m, 4H, NCH₂CH₂NH), 1.49 (bs, 9H, C(CH₃)₃). MS (Cl/NH₃) found 534.2, expected for C₂₈H₃₀N₅O₆ 532.5.

### Example 25

### Triethylammonium [{2-[Acridine-9-carbonyl)-amino]-acetyl}-(2-tert-butoxycarbonylamino-ethyl)-amino]-acetate (Compound 9a)

[{2-[Acridine-9-carbonylyamino]-acetyl}-(2-tert-butoxycarbonylamino-ethyl)-amino]-acetic acid methyl ester (0.096 g, 0.19 mmol) was treated with 0.2 ml of a concentrated solution of NaOH in 6 ml of EtOH:H₂O (1:2) solution at room temperature for 2 h. The reaction mixture was concentrated to dryness. The residue was dissolved in the minimum amount of water and acidified to pH=2. The resulting residue was purified on silica gel (CH₂Cl₂:CH₃OH/90:10) containing 1% of triethylamine yielded the desired compound (37 mg, 34%) as a triethylammonium salt. ¹H NMR [MeOD δ, ppm]: ]: 8.44 (m, 2H), 8.20 (m, 2H), 7.91 (m, 2H), 7.68 (m, 2H), 4.61 (s, 2H, (CH₂CON), 4.47 (s, 2H, (CH₂COO), 3.62 and 3.33 (m, 4H, NCH₂CH₂NH), 1.49 (bs, 9H, C(CH₃)₃). MS (Cl/NH₃) found 481.3, expected for C₂₅H₂₈N₄O₆ 480.7.

### Example 26

### Triethylammonium [(2-tertbutoxycarbonvlamino-ethyl)-{2-[(10H-indolo[3,2-b]quinoline-11-carbonyl)-amino]-acetyl}-amino)-acetate (Compound 9b)

[(2-tert-Butoxycarbonylamino-ethyl)-{2-[10H-indolo[3,2-b]quinoline-11-carbonyl)amino]-acetyl}-amino)acetic acid methyl ester (0.057 g, 0.1 mmol) was treated with 0.1 ml of a concentrated solution of NaOH in 6 ml of EtOH:H₂O (1:2) solution at room temperature for 2 h. The reaction mixture was concentrated to dryness. The residue was dissolved in the minimum amount of water and acidified to pH=2. The resulting residue was purified on silica gel (CH₂Cl₂:CH₃OH/90:10) containing 1% of triethylamine yielded the desired compound (13 mg, 20%) as a triethylammonium salt. ¹H NMR [MeOD δ, ppm]: ]: 8.48 (m, 1H), 8.35 (m, 1H), 8.29 (m, 1H), 7.75 (m, 1H), 7.68 (m, 2H), 7.60 (m, 1H), 7.35 (m, 1H), 4.63 (s, 2H, (CH₂CON), 4.48 (s, 2H, (CH₂COO), 3.62 and 3.33 (m, 4H, NCH₂CH₂NH), 1.49 (bs, 9H, C(CH₃)₃). MS (Cl/NH₃) found 520.2, expected for C₂₇H₂₉N₅O₆ 519.5.

### Example 27

### O-{2-N-(Acridine-9-carbamoyl)-(1-hydroxybut-3-yl)} and O-{2-N-(10H-indolo[3,2-d]quinoline-11-carbamoyl)-3-hydroxybut-1-yl) phosphate (1-Act-p-Qut-3)

Controlled pore glass loaded with N-(3-hydroxy-1-(4,4′-dimethoxytrityl)oxybutan-2-yl)-10H-indolo[3,2-d]quinoline-11-carboxamide (compound 3a) prepared as described in example 7 (1 μmol) was treated with a 3% trichloroacetic acid solution in dichloromethane. After treatment and washing with acetonitrile N-(3-(N,N-diisopropylamino-2-cyanoethoxyphosphynyl)oxy-1-(4,4′-dimethoxytrityl)oxybutan-2-yl)acridine-9-carboxamide (compound 4b, 20 μmol, 16.5 mg) dissolved in 200 μl of anhydrous ACN and tetrazole (80 mmols, 100 μl of a 0.8 M solution in acetonitrile) were added under argon atmosphere. After 5 minutes, the solution was removed and the glass support was washed with acetonitrile. Oxidation of the resulting phosphite-triester to phosphate-triester was done with an iodine solution (I₂ in water/pyridine/THF 2/20/80), during 2 minutes. Additional acetonitrile and dichloromethane washes were performed. Finally, deprotection of the dimethoxytrityl group from the support was done using trichloroacetic acid 3%. Dimer 1-Act-p-Qut-3 was released from the resin using 32% ammonia solution during 1.5 h at 55° C. The ammonia mixture was filtered and concentrated to dryness. The residue was passed over a Dowex 50×4 (Na⁺ form) column to exchange ammonia ions for Na⁺ ions. Fractions containing the desired product were analysed by HPLC, UV and MS.

HPLC solutions were solvent A: 5% acetonitrile in 100 mM triethylammonium acetate, pH 6.5 and solvent B: 70% acetonitrile in 100 mM triethylammonium acetate (pH 6.5). Column: PRP-1(Hamilton) 250×10 mm. Flow rate 3 ml/min linear gradient from 15 to 80 in B. HPLC chromatogram shows an unique peak at 12.7 min. MS (MALDI-TOF): found 766.5 [M+2Na⁺], expected for C₃₈H₃₆N₅O₈P 721.7. Yield: 45%.

### Example 28

### O-{2-N-(10H-indolo[3,2-d]quinoline-11-carbamoyl)-(1-hydroxybut-3-yl)} and O-(2-N-{10H-indolo[3,2-d]quinoline-11-carbamoyl}-3-hydroxybut-1-yl)phosphate (1-Qut-P-Qut-3)

N-(3-(N,N-diisopropylamino-2-cyanoethoxyphosphynyl)oxy-1-(4,4′-dimethoxytrityl)oxybutan-2-yl)-10H-indolo[3,2-d]quinoline-11-carboxamide (compound 4a) was coupled to controlled pore glass loaded with N-(3-hydroxy-1-(4,4′-dimethoxytrityl)oxybutan-2-yl)-10H-indolo[3,2-d]quinoline-11-carboxamide (compound 3a) prepared as described in example 7 (1 μmol) as described in example 39. After the exchange sodium column, the desired product was analysed as above. HPLC chromatogram of the product shows the presence of one major peak with a retention time of 14.4 min (see HPLC conditions in example 27). MS (MALDI-TOF): found 761.3 [M+H⁺], 783.3 [M−H⁺+Na⁺], expected for C₄₀H₃₇N₆O₈P 760.7. Yield: 12%.

### Example 29

### O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-(2-N-{10H-indolo[3,2-d]quinoline-11-carbamoyl}-3-hydroxybut-1-yl) (1-Act-p-Qut-P-Qut-3)

Trimer 1-Act-p-Qut-p-Qut-3 was obtained by the addition of the monomer N-(3-(N,N-diisopropylamino-2-cyanoethoxyphosphynyl)oxy-1-(4,4′-dimethoxytrityl)oxybutan-2-yl)acridine-9-carboxamide (compound 4a) to 1-Qut-p-Qut-3 dimer described in example 40. The coupling was performed as described in example 39. After ammonia deprotection the desired compound was purified by HPLC. Retention time in HPLC (see conditions in example 27) 15.3 min. MS (MALDI-TOF): found 1139.8 [M+H⁺] 1117.7 [M−OH⁻+H⁺], expected for C₅₈H₅₄N₈O₁₃P₂ 1133.0. Yield: 7%.

### Example 30

### O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-3-hydroxybut-1-yl} (1-Act-p-Act-p-Act-p-Act-p-Act-p-Act-3)

N-(3-(N,N-diisopropylamino-2-cyanoethoxyphosphynyl)oxy-1-(4,4′-dimethoxytrityl)oxybutan-2-yl)acridine-9-carboxamide (compound 4b) was reacted to a glass support loaded with N-(3-hydroxy-1-(4,4′-dimethoxytrityl)oxybutan-2-yl)acridine-9-carboxamide (compound 3b) prepared as described in example 9. The coupling was performed as described in example 39. The addition of N-(3-(N,N-diisopropylamino-2-cyanoethoxyphosphynyl)oxy-1-(4,4′-dimethoxytrityl)oxybutan-2-yl)acridine-9-carboxamide (compound 4b) was repeated 5 times in order to obtain the hexamer. After ammonia deprotection the desired compound was purified by HPLC. Retention time in HPLC (see conditions in example 27) 11.6 min. MS (MALDI-TOF): found 2172.3 [M+H⁺], expected for C₁₀₈H₁₀₃N₁₂O₂₈P₅ 2171.8. Yield: 1.4 mg (60%).

### Example 31

### O-{2-N-(Acridine-9-carbamoyl)-(1-hydroxybut-3-yl)} and O-{3-(5-methyl-5H-indolo[3,2b]quinolin-11-ylamino-(S)-(2-hydroxyprop-3-yl} phosphate (1-Act-p-Cra-2)

N-(3-(N,N-diisopropylamino-2-cyanoethoxyphosphynyl)oxy-1-(4,4′-dimethoxytrityl)oxybutan-2-yl)acridine-9-carboxamide (compound 4b) was coupled to controlled pore glass loaded with (S)-3-(5-Methyl-5H-indolo[3,2b]quinolin-11-ylamino)-1-(4,4′-dimethoxytrityl)oxypropane-2-ol (compound 6c) prepared as described in example 34 (1 μmol). The coupling was performed as described in example 39. After ammonia deprotection the desired compound was purified by HPLC. Retention time in HPLC (see conditions in example 27) 13.0 min. MALDI-TOF mass spectrometry: found 694.64 [M+H⁺], expected for C₃₇H₃₆N₅O₇P 693.65. Yield: 13%.

### Example 32

### O-{2-N-(Acridine-9-carbamoyl)-(1-hydroxybut-3-yl)} and O-}3-(5-methyl-5H-indolo[3,2b]quinolin-11-ylamino-(S)-(2-hydroxyprop-3-yl} phosphorotioate (1-Act-ps-Cra-2)

N-(3-(N,N-diisopropylamino-2-cyanoethoxyphosphynyl)oxy-1-(4,4′-dimethoxytrityl)oxybutan-2-yl)acridine-9-carboxamide (compound 4b) was coupled to controlled pore glass loaded with (S)-3-(5-Methyl-5H-indolo[3,2b]quinolin-11-ylamino)-1-(4,4′-dimethoxytrityl)oxypropane-2-ol (compound 6c) prepared as described in example 34 (1 μmol). The coupling was performed as described in example 39 except that in this case sulfurization was performed with 1 ml of a solution of 10 mg of 3H-1,2-benzodithiol-3-one 1,1-dioxide in acetonitrile (1 min) (c.f. R. P. Iyer et al., “The automated synthesis of sulfur-containing oligodeoxyribonucleotides using 3H-1,2-benzodithiol-3-one 1,1-dioxide as a sulfur-transfer reagent” J. Org. Chem. 1990, vol. 55, pp 4693-4699) instead of oxidation with iodine solution. After ammonia deprotection the desired compound was purified by HPLC. Retention time in HPLC (see conditions in example 27) 15.0 and 15.5 min (2 disatereoisomers). MS (MALDI-TOF): found 708.4 [M], expected for C₃₇H₃₆N₅O₆PS 709.63. Yield: 20%.

### Example 33

### O-[(R)-3-(Acridin-9-ylamino)-1-hydroxy-propane-2-yl]-phosphate (2-1) O-{2-N-(acridine-9-ylamino)-2-oxyprop-1-yl} phosphate(2-4) 4-hydroxybutirame (1-Aca-p-Aca-p-butvramide)

4-Trityl-4-hydroxybutanoic acid was reacted with polystyrene loaded with Rink-amide linker (c.f. H. Rink, “Solid-phase synthesis of protected peptide fragments using a trialkoxy-diphenyl-methylester resin” Tetrahedron Lett., 1987, vol 28, pp 3787). The resulting support was used for the assembly of the s. O-[(R)-3-(acridin-9-ylamino)-1-(4,4′-dimethoxytrityl)oxypropane-2-yl]-N,N-diisopropyl-amino-2-cyanoethoxy phosphoramidite (compound 7a) was reacted to the support loaded with 4-trityl-hydroxybutanoic Rink amide. The coupling was performed as described in example 27. After the assembly of the dimer, the supports were treated with 0.5 M DBU solution for 1 min, washed with acetonitrile and treated with a solution containing 95% trifluoroacetic acid and 5% water for 4 hr at room temperature. Retention time in HPLC (see conditions in example 27) 5.8 min. MS (MALDI-TOF): found 764.6 [M+H⁺], expected for C₃₆H₃₉N₅O₁₀P₂ 763.6. Yield: 25%.

### Example 34

### O-{2-N-(2-phenylquinoline-4-carbamoyl-1-hydroxybutan-3-yl)} and O-{2-N-(acridine-9-carbamoyl)-3-hydroxybut-1-yl} phosphate (1-Pht-p-Act-3)

N-((2S, 3R)-3-(N,N-diisopropylamino-2-cyanoethoxyphosphinyl)oxy-1-(dimethoxytrityloxy)-butan-2-yl)-2-phenylquinoline-4-carboxamide (compound 4e, example 15) was reacted with a glass support loaded with N-(3-hydroxy-1-(4,4′-dimethoxytrityl)oxybutan-2-yl)acridine-9-carboxamide (compound 3b) prepared as described in example 9. The coupling was performed as described in example 27. After ammonia deprotection the desired compound was purified by HPLC. Retention time in HPLC (see conditions in example 27) 14.5 min. MS (MALDI-TOF): found 707.8 [M], expected for C₃₈H₃₇N₄O₅P 708.7. Yield: 22%.

### Example 35

### O-{2-N-(2-phenylquinoline-4-carbamoyl-1-hydroxybutan-3-yl)} and O-{2-N-(2-phenylquinoline-4-carbamoyl-3-hydroxybutan-1-yl)} phosphate (1-Pht-p-Pht-3)

N-((2S, 3R)-3-(N,N-diisopropylamino-2-cyanoethoxyphosphinyl)oxy-1-(dimethoxytrityloxy)-butan-2-yl)-2-phenylquinoline-4-carboxamide (compound 4e, example 15) was reacted with a glass support loaded with N-((2S, 3R)-3-hydroxy-1-(dimethoxytrityloxy)-butan-2-yl)-2-phenylquinoline-4-carboxamide (compound 3e) prepared as described in example 14. The coupling was performed as described in example 27. After ammonia deprotection the desired compound was purified by HPLC. Retention time in HPLC (see conditions in example 27) 19.1 min. MS (MALDI-TOF): found 733.7 [M], expected for C₄₀H₃₉N₄O₈P 734.7. Yield: 18%.

### Example 36

### O-{2-N-(acridine-9-carbamoyl)-1-hydroxybut-3-yl} and O-{2-N-(2-phenylquinoline-4-carbamoyl-3-hydroxybutan-1-yl)} phosphate (1-Act-p-Pht-3)

N-(3-(N,N-diisopropylamino-2-cyanoethoxyphosphinyl)oxy-1-(4,4′-dimethoxytrityl)oxybutan-2-yl)acridine-9-carboxamide (compound 4b, example 10) was reacted with a glass support loaded with N-((2S, 3R)-3-hydroxy-1-(dimethoxytrityloxy)-butan-2-yl)-2-phenylquinoline-4-carboxamide (compound 3e) prepared as described in example 14. The coupling was performed as described in example 27. After ammonia deprotection the desired compound was purified by HPLC. Retention time in HPLC (see conditions in example 27) 14.9 min. MS (MALDI-TOF): found 707.6 [M], expected for C₃₈H₃₇N₄O₈P 708.7. Yield: 24%.

### Example 37

### O-{2-N-(2-phenylquinoline-4-carbamoyl-1-hydroxybutan-3-yl)} and O-(2-N-{10H-indolo[3,2-d]quinoline-11-carbamoyl}-3-hydroxybut-1-yl) (1-Pht-p-Qut-3)

N-((2S, 3R)-3-(N,N-diisopropylamino-2-cyanoethoxyphosphinyl)oxy-1-(dimethoxytrityloxy)-butan-2-yl)-2-phenylquinoline-4-carboxamide (compound 4e, example 15) was reacted was reacted with a glass support loaded with N-(3-hydroxy-1-(4,4′-dimethoxytrityl)oxybutan-2-yl)-10H-indolo[3,2-d]quinoline-11-carboxamide (compound 3a, example 7, 1 μmol). The coupling was performed as described in example 27. After ammonia deprotection the desired compound was purified by HPLC. Retention time in HPLC (see conditions in example 27) 16.8 min. MS (MALDI-TOF): found 746.6 [M], expected for C₄₀H₃₈N₅O₈P 747.7. Yield: 24%.

### Example 38

### O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carboxamido)acetamidol-(3-hydroxybut-1-yl)} (1-Act-p-Qut-p-Aqt-3)

N-(3-(N,N-diisopropylamino-2-cyanoethoxyphosphynyl)oxy-1-(4,4′-dimethoxytrityl)oxybutan-2-yl)-10H-indolo[3,2-d]quinoline-11-carboxamide (compound 4a) was coupled to controlled pore glass loaded with N-(3-hydroxy-1-(4,4′-dimethoxytrityl)oxybutan-2-ylcarbamoyl)methyl)acridine-9-carboxamide (compound 3d, 1 mol, example 12) as described in example 27. After coupling, the DMT group was removed and the resulting support was reacted with N-(3-(N,N-diisopropylamino-2-cyanoethoxyphosphinyl)oxy-1-(4,4′-dimethoxytrityl)oxybutan-2-yl)acridine-9-carboxamide (compound 4b, example 10). The coupling was performed as described in example 40. After ammonia deprotection the desired compound was purified by HPLC. Retention time in HPLC (see conditions in example 27) 12.3 min. MS (MALDI-TOF): found 1152.2 [M+H⁺], expected for C₅₈H₅₆N₈O₁₄P₂ 1151.0. Yield: 20%.

### Example 39

### (2S, 3R)-2-((9H-Fluoren-9-yl)methyloxycarbonyl)amino)butane-1-(4,4′-dimethoxytrityloxy)butan-3-ol

(2S, 3R)-2-((9H-Fluoren-9-yl)methyloxycarbonyl)amino)butane-1,3-diol (0.4 g, 1.22 mmol) was reacted with 4,4-dimethoxytrityl chloride (0.5 g, 1.46 mmol) and N,N-dimethylaminopiridine (9.4 mg) in 20 ml of pyridine. After 3 hr of magnetic stirring at room temperature, 83 mg of 4,4′-dimethoxytrityl chloride (0.24 mmol) were added and the reaction was stirred for 1 hour. After, the reaction was stopped with the addition of 1 ml of methanol and the mixture was concentrated to dryness. The residue was dissolved in CH₂Cl₂ and washed with brine and dried. The resulting product was purified by chromatography on silica gel (CH₂Cl₂) yielding 0.59 g of the desired DMT, Fmoc-protected derivative (93%) as a foam. TLC (5% methanol in CH₂Cl₂) Rf 0.68. ¹H-NMR [CDCl₃, δ, ppm]: 7.8-7.2 (m, 17H, Ar Fluorenyl, DMT), 6.8 (dd, 4H, Ar DMT), 5.4 (d, 2H), 4.4 (m, 1H), 4.3 (m, 1H), 4.2 (m, 1H), 4.1 (m, 1H), 3.7 (s, 6H, OMe DMT), 3.6 (m, 1H), 3.45 (m, 1H), 3.27 (m, 1H), 2.8 (m, 1H), 1.16 (d, 3H, CH₃).

### Example 40

### (2S, 3R)-2-((9H-Fluoren-9-yl)methyloxycarbonyl)amino)butane-1-(4,4′-dimethoxytrityloxy)butan-3-yl N,N-diisopropylamino-2-cyanoethyl phosphoramidite

(2S, 3R)-2-((9H-Fluoren-9-yl)methyloxycarbonyl)amino)butane-1-(4,4′-dimethoxytrityloxy)butan-3-ol (170 mg, 0.27 mmol) was dried by coevaporation of dry acetonitrile and dissolved in 5 ml of dry dichloromethane. N,N-Diisopropylethylamine (0.14 ml, 0.8 mmol) was added and the mixture was purged with argon and cooled with an ice-water bath. O-2-Cyanoethyl-N,N-diisopropyl-chlorophosphoramidite (0.090 ml, 0.40 mmol) was added dropwise during 10 min with continuous stirring. After the addition of the chlorophosphine, the mixture was allowed to warm to room temperature and stirred for 1 hr. The reaction was stopped by addition of 0.5 ml of water and the mixture was concentrated to dryness. The residue was dissolved in CH₂Cl₂ containing 1% of triethylamine and washed with brine and dried. The resulting product was purified by chromatography on silica gel (hexane/ethyl acetate (3:1)+1% triethylamine) yielding 170 mg of the desired phosphoramidite (76%) as a foam. ¹H-NMR [CDCl₃, δ, ppm]: 7.8-7.2 (m, 17H, Ar Fluorenyl, DMT), 6.8 (dd, 4H, Ar DMT), 5.0 (d, 2H), 4.4 (m, 2H), 4.2 (m, 2H), 3.8 (m, 1H), 3.73 (s, 6H, OMe DMT), 3.4 (m, 2H), 3.2 (m, 2H), 2.55 (t, 2H), 1.24-0.95 (m, 15H, 5CH₃). ³¹P-NMR [CDCl₃, δ, ppm]: 148.87 and 148.63 (two diastereoisomers).

### Example 41

### O-{2-N-(10H-indolo[3,2-d]quinoline-11-carbamoyl)-3-oxybut-1-yl} phosphate (3-1) O-{3-(5-methyl-5H-indolo[3,2-b]quinolin-11-ylamino-(S)-(2hydroxylprop-3-yl) (1-Qut-p-Cra-2)

(2S, 3R)-2-((9H-Fluoren-9-yl)methyloxycarbonyl)amino)butane-1-(4,4′-dimethoxytrityloxy)butan-3-yl N,N-diisopropylamino-2-cyanoethyl phosphoramidite (described in example 59) was reacted with a glass support loaded with (S)-3-(5-Methyl-5H-indolo[3,2b]quinolin-11-ylamino)-1-(4,4′-dimethoxytrityl)oxypropane-2-ol (compound 6b, example 34, 0.5 μmol). The coupling was performed as described in example 27. After the coupling and oxidation, the support was treated with 20% piperidine in DMF for 5 min to remove the Fmoc group. Coupling of 10H-indolo[3,2-d]quinoline-11-carboxylic acid (compound 1a) to the support was performed using 6.5 mg of compound 1a (0.025 mmol), 13 mg of PyBOP (0.0025 mmol) and 0.0087 ml of diisopropylethylamine for 90 min at room temperature. Then the support was treated with 3% trichloroacetic acid in CH₂Cl₂ to remove the DMT group and finally the resulting support was treated with concentrated ammonia. After ammonia deprotection the desired compound was purified by HPLC. Retention time in HPLC (see conditions than in example 27) 14.4 min. MS (MALDI-TOF): found 733 [M+H⁺], expected for C₃₉H₃₇N₆O₇P 732.7 Yield: 22%.

### Example 42

### O-{2-N-(10H-indolo[3,2-d]quinoline-11-carbamoyl)-3-oxybut-1-yl} phosphate (3-1) O-{2-N-(10H-indolo[3,2-d]quinoline-11-carbamoyl)-3-oxybut-1-yl} phosphate (3-1) O-(2-N-{10H-indolo[3,2-d]quinoline-11-carbamoyl}-3-hydroxybut-1-yl) (1-Qut-p-Qut-P-Qut-3)

The trimer was prepared as described in example 38. Retention time in HPLC (see conditions in example 27) 15.3 min. MS (MALDI-TOF): found 1238 [M+3Na⁺], expected for C₆₀H₅₅N₉O₁₃P₂ 1172.0. Yield: 11%.

### Example 43

### O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(3-hydroxybut-1-yl)} (1-Act-p-Act-p-Act-3)

The trimer was prepared as described in example 38. Retention time in HPLC (see conditions in example 27) 11.3 min. MS (MALDI-TOF): found 1077.1 [M+Na⁺], expected for C₅₄H₅₂N₆O₁₃P₂ 1154.9. Yield: 42%.

### Example 44

### O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carbamoyl)-(3-hydroxybut-1-yl)} (1-Act-p-Qut-p-Act-3)

The trimer was prepared as described in example 38. Retention time in HPLC (see conditions in example 27) 11.3 min. MS (MALDI-TOF): found 1093.9 [M+2H⁺], expected for C₅₆H₅₃N₇O₁₃P₂ 1093.9. Yield: 25%.

### Example 45

### O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(5H-indolo[2,3-b]quinolin-5-yl)acetamido-3-hydroxybut-1-yl) (1-Act-p-Qut-p-Nct-3)

The trimer was prepared as described in example 38. Retention time in HPLC (see conditions in example 27) 16.8 min. MS (MALDI-TOF): found 1146 [M], expected for C₅₉H₅₅N₈O₁₃P₂ 1146.0. Yield: 12%.

### Example 46

### Acetyl-{2-(Acridine-9-carbonyl)-amino]-acetyl}-(2-aminoethyl)-glycyl-{2-(Acridine-9-carbonyl)-amino]-acetyl}-(2-aminoethyl)-N-6-hydroxyhexyl glycynamide (Ac-Aqp-Aqp-NH-hexyl-OH)

Polyethylene-polystyrene support containing aminohexylsuccinil linker (50 mg, 7.5 μmol) was reacted triethylammonium [(2-[acridine-9-carbonylyamino]-acethyl}-(2-tertbutoxycarbonylamino-ethyl)-amino]-acetate prepared as described above. For this purpose, triethylammonium [(2-[acridine-9-carbonyl)-amino]-acethyl}-(2-tertbutoxycarbonylamino-ethyl)-amino]-acetate was dissolved in 150 μl of DMF, then diisopropylamine (19.6 μl, 0.11 mmols) was added and {O-(7-azabenzotriazol-1-yl]-1,1,3,3-tetramethyluronium hexafluorophosphate, HATU (12.8 mg, 0.033 mmols). The reaction mixture was left at room temperature for 1-2 min and the resulting solution was added to the support. Coupling reaction was maintained for 1 hour at room temperature. After this period the support was filtered and washed with DMF and DCM. Deprotection of the BOC group was done with a solution of 50% trifluoroacetic acid in dichloromethane and 5% cresol (20 min). After the assembly of the dimer, the solid support was capped with acetic anhydride and N-diisopropylethylamine in DMF (1:1.9:10) for 10 min at room temperature. Finally, the support was treated with 32% ammonia solution for 2 h at 55° C. The resulting solution was filtered and concentrated. HPLC analysis showed a major peak that had the expected molecular weight. MS (MALDI): found 884.4, expected 885.9 for C₄₈H₅₅N₉O₈.

### Example 47

### Acetyl-{2-(Acridine-9-carbonyl)-amino]-acetyl}-(2-aminoethyl)-glycyl-{2-(Acridine-9-carbonyl)-amino]-acetyl}-(2-aminoethyl)-glycyl {2-(Acridine-9-carbonyl)-amino]-acetyl}-(2-aminoethyl)-N-6-hydroxyhexyl glycynamide (Ac-Aqp-Aqp-Aqp-NH-hexyl-OH)

The oligomer was synthesized as described in example 54 but with an extra acridine monomer coupling. Acetylation also was performed in the same conditions as described above for the synthesis of the dimer. HPLC chromatogram reveals one major peak and two small secondary peaks. The major peak with tr=13.9 min had the expected mass. MS (MALDI): found 1246.4, expected 1249.3 for C₆₈H₇₄N₁₃O₁₁. One small secondary peaks was characterized as the corresponding acridine dimer (MS (MALDI): found 884.3, expected 885.9 for C₄₈H₅₅N₉O₈).

### Example 48

### Acetyl-{2-N-(10H-indolo[3,2-d]quinoline-11-carbonyl)-amino]-acetyl}-(2-aminoethyl)-glycyl-{2-N-(10H-indolo[3,2-d]quinoline-11-carbonyl)-amino]-acetyl}-(2-aminoethyl)-N-6-hydroxyhexyl glycynamide (Ac-Qgp-Qgp-NH-hexyl-OH)

The oligomer was synthesized as described in example 54 but using triethylammonium [(2-tertbutoxycarbonylamino-ethyl)-{2-[(10H-indolo[3,2-b]quinoline-11-carbonyl)-amino]-acetyl}-amino)-acetate as monomer, diisopropylcarbodiimide as coupling agent and 1-hydroxybenzotriazol as catalyst. Retention time in HPLC (see conditions in example 39) 16.2 min. MS (MALDI): found 962.6 [M], 984.6 [M+Na⁺], expected for C₅₂H₅₇N₁₁O₈ 964.0.

### Example 49

### N-[2-(Acridine-9-carboxamide)[-4-N-[2-(acridine-9-carboxamide)prolinamide

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and acridine-9-carboxylic acid (3 eq) was coupled to the resin using TBTU (3 eq) and DIEA (6 eq) for the coupling. The resin was then treated with 20% piperidine in DMF and the N-terminal and the second fluorophore (acridine-9-carboxylic acid (3 eq)) was introduced using the same coupling reagents described above. The final compound was cleaved with anhydrous HF at 0° C. during 1h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. HPLC solutions were solvent A: H₂O containing 0.045% of TFA. Solvent B: acetonitrile containing 0.036% of TFA. Column: reverse-phase Symmetry C₁₈ (150×4.6 mm) 5 m, with UV detection at 220 nm. Flow rate 1 ml/min linear gradient from 0 to 50 in B. HPLC chromatogram shows the compound 78% pure at retention time of 8.3 min. MS (electrospray): found 598.33 [M+H⁺], expected for C₃₅H₂₈N₆O₄ 596.63.

### Example 50

### N-[2-(Acridine-9-carboxamide)acetyl]-4-N-acetamidoprolinamide

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and acridine-9-carboxylic acid (3 eq) was coupled to the resin using TBTU (3 eq) and DIEA (6 eq) for the coupling. The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 68): 6.7 min, 95%. MS (electrospray): found 434.24 [M+H⁺], expected for C₂₃H₂₃N₅O₄ 433.46.

### Example 51

### N-{2-(10H-indolo[3,2-b]quinoline-11-carboxamide)acetyl}-4-N-acetamidoprolinamide

This compound was synthesized as example 69, changing the fluorophore for 10H-indolo[3,2-b]quinoline-11-carboxylic acid (3 eq). The desired product was characterized by UV-spectra and mass spectrometry. Retention time and purity in the HPLC (see conditions in example 68): 8.6 min, 96%. MS (electrospray): found 473.29 [M+H⁺], expected for C₂₅H₂₄N₆O₄ 472.50.

### Example 52

### N⁴-Acetyl-4-amino-{2-(acridine-9-carboxamide)acetyl}prolinyl-4-amino-{2-(acridine-9-carboxamide)acetyl}prolinamide

Boc-Amp(Fmoc)-OH (3 eq) was coupled to the MBHA resin using DIPCDI (3 eq) and HOBt (3 eq) as coupling reagents. The resin was then treated with 20% piperidine in DMF and the second molecule of Boc-Amp(Fmoc)-OH (3 eq) was coupled using the same coupling reagents. Then Boc protecting groups were removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and acridine-9-carboxylic acid (6 eq) was coupled to the resin using TBTU (6 eq) and DIEA (12 eq) for the coupling. The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 68): 7.6 min, 75%. MS (electrospray): found 808.36 [M+H⁺], expected for C₄₄H₄₁N₉O₇ 807.85.

### Example 53

### N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinamide

This compound was synthesized as example 71, changing the fluorophore for 10H-indolo[3,2-b]quinoline-11-carboxylic acid (6 eq). The desired product was characterized by UV-spectra and mass spectrometry. Retention time and purity in the HPLC (see conditions in example 68): 9.5 min, 81%. MS (electrospray): found 886.48 [M+H⁺], expected for C₄₈H₄₃N₁₁O₇ 885.92.

### Example 54

### 10H-Indolo[3,2-b]quinoline-11-carboxylic acid {2-[4-acetylamino-2-(1-{2-[(acridine-9-carbonyl)-amino]-acetyl}-5-carbamoyl-pyrrolidin-3-ylcarbamoyl)-pyrrolidin-1-yl]-2-oxo-ethyl}-amide

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and acridine-9-carboxylic acid (3 eq) was coupled to the resin using TBTU (3 eq) and DIEA (3 eq). The resin was then treated with 20% piperidine in DMF and Boc-Amp(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and 10H-indolo[3,2-b]quinoline-11-carboxylic acid (3 eq) was coupled to the resin using TBTU (3 eq) and DIEA (6 eq). The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 68): 8.5 min, 86%. MS (electrospray): found 847.30 [M+H⁺], expected for C₄₆H₄₂N₁₀O₇ 846.89.

### Example 55

### 10H-Indolo[3,2-b]quinoline-11-carboxylic acid (2-{4-[4(4-acetylamino-1-{2-[(acridine-9-carbonyl)-amino]-acetyl}-pyrrolidine-2-carbonyl)-amino]-2-carbamoyl-pyrrolidin-1-yl}-2-oxo-ethyl)-amide

This compound was synthesized following the same protocol described for example 73, but replacing the first fluorophore for 10H-indolo[3,2-b]quinoline-11-carboxylic acid (3 eq) and the second one for acridine-9-carboxylic acid (3 eq). The desired product was characterized by UV-spectra and mass spectrometry. Retention time and purity in the HPLC (see conditions in example 68): 8.7 min, 88%. MS (electrospray): found 847.36 [M+H⁺], expected for C₄₆H₄₂N₁₀O₇ 846.89.

### Example 56

### N⁴-Acetyl-4-amino-{2-(acridine-9-carboxamide)acetyl}prolinyl-4-amino-{2-(acridine-9-carboxamide)acetyl}prolinyl-4-amino-{2-(acridine-9-carboxamide)acetyl}prolinamide

Boc-Amp(Fmoc)-OH (3 eq) was coupled to the MBHA resin using DIPCDI (3 eq) and HOBt (3 eq) as coupling reagents. The resin was then treated with 20% piperidine in DMF and the second molecule of Boc-Amp(Fmoc)-OH (3 eq) was coupled using the same coupling reagents. The resin was treated again with 20% piperidine in DMF and the third Boc-Amp(Fmoc)-OH (3 eq) was coupled using the same coupling reagents. Then Boc protecting groups were removed with 40% TFA in DCM, the resin was treated with 5% DIEA in DCM and the spacer (Boc-Gly-OH, 15 eq) was introduced at this α-amino position using DIPCDI (15 eq) and HOBt (15 eq) as coupling reagents. After that, Boc group was removed and acridine-9-carboxylic acid (9 eq) was coupled to the resin using TBTU (9 eq) and DIEA (18 eq) for the coupling. The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 68): 8.1 min, 56%. MS (electrospray): found 1182.65 [M+H⁺], expected for C₆₅H₅₉N₁₃O₁₀ 1182.25.

### Example 57

### N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-{2-(10H-indolo[3,2-b]quinoline-11-carboxamide)acetyl}prolinamide

This compound was synthesized following the same protocol described for example 56, but replacing the fluorophore for 10H-indolo[3,2-b]quinoline-11-carboxylic acid (9 eq). The desired product was characterized by UV-spectra and mass spectrometry. Retention time and purity in the HPLC (see conditions in example 68): 10.1 min, 87%. MS (electrospray): found 1299.66 [M+H⁺], expected for C₇₁H₆₂N₁₆O₁₀ 1299.35.

### Example 58

### N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(acridine-9-carboxamide)acetyl}prolinamide (Ac-Aqr-Qqr-Aqr-NH₂)

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the first fluorophore (acridine-9-carboxylic acid (3 eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the second fluorophore (10H-indolo[3,2-b]quinoline-11-carboxylic acid (3 eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the third fluorophore (acridine-9-carboxylic acid (3 eq)) was coupled to the resin using TBTU (3 eq) and DIEA (6 eq). The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 68): 8.5 min, 75%. MS (electrospray): found 1222.56 [M+H⁺], expected for C₆₇H₆₀N₁₄O₁₀ 1221.28.

### Example 59

### N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-4-amino-{2-(acridine-9-carboxamide)acetyl}prolinamide (Ac-Qqr-Qqr-Aqr-NH₂)

This compound was synthesized following the same protocol described for example 58, but replacing the third fluorophore for 10H-indolo[3,2-b]quinoline-11-carboxylic acid (3 eq). The desired product was characterized by UV-spectra and mass spectrometry. Retention time and purity in the HPLC (see conditions in example 68): 9.3 min, 71%. MS (electrospray): found 1182.65 [M+H⁺], expected for C₆₉H₆₁N₁₅O₁₀ 1260.32.

### Example 60

### N⁴-Acetyl-4-amino-{2-(10H-indolo[3,2-b]quinoline-11-carboxamide)acetyl}prolinyl-4-amino-{2-(acridine-9-carboxamide)acetyl}prolinyl-4-amino-{2-(acridine-9-carboxamide)acetyl}prolinamide (Ac-Qgr-Agr-Agr-NH₂)

This compound was synthesized following the same protocol described for example 58, but replacing the second fluorophore for acridine-9-carboxylic acid (3 eq) and the third one for 10H-indolo[3,2-b]quinoline-11-carboxylic acid (3 eq). The desired product was characterized by UV-spectra and mass spectrometry. Retention time and purity in the HPLC (see conditions in example 68): 8.8 min, 57%. MS (electrospray): found 1222.61 [M+H⁺], expected for C₆₇H₆₀N₁₄O₁₀ 1221.28.

### Example 61

### N⁴-Acetyl-4-amino-{2-(10H-indolo[3,2-b]quinoline-11-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide (Ac-Qgr-Agr-Qgr-NH₂)

This compound was synthesized following the same protocol described for example 58, but replacing the first fluorophore for 10H-indolo[3,2-b]quinoline-11-carboxylic acid (3 eq), the second one for acridine-9-carboxylic acid (3 eq) and the third one for 10H-indolo[3,2-b]quinoline-11-carboxylic acid (3 eq). The desired product was characterized by UV-spectra and mass spectrometry. Retention time and purity in the HPLC (see conditions in example 68): 9.8 min, 84%. MS (electrospray): found 1261.63 [M+H⁺], expected for C₆₉H₆₁N₁₅O₁₀ 1260.32.

### Example 62

### N⁴-Acetyl-4-amino-{2-(acridine-9-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide (Ac-Agr-Agr-Qgr-NH₂)

This compound was synthesized following the same protocol described for example 58, but replacing the first fluorophore for 10H-indolo[3,2-b]quinoline-11-carboxylic acid (3 eq) and the second one for acridine-9-carboxylic acid (3 eq). The desired product was characterized by UV-spectra and mass spectrometry. Retention time and purity in the HPLC (see conditions in example 68): 9.5 min, 79%. MS (electrospray): found 1221.69 [M+H⁺], expected for C₆₇H₆₀N₁₄O₁₀ 1221.28.

### Example 63

### N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(10H-indolo[3,2-b]quinoline-11-carboxamide)acetyl}prolinamide (Ac-Agr-Qgr-Qgr-NH₂)

This compound was synthesized following the same protocol described for example 58, but replacing the first fluorophore for 10H-indolo[3,2-b]quinoline-11-carboxylic acid (3 eq). The desired product was characterized by UV-spectra and mass spectrometry. Retention time and purity in the HPLC (see conditions in example 68): 8.9 min, 62%. MS (electrospray): found 1260.63 [M+H⁺], expected for C₆₉H₆₁N₁₅O₁₀ 1260.32.

### Example 64

### N⁴-Acetyl-4-amino-{2-(10H-indolo[3,2-b]quinoline-11-carboxamide)acetyl}prolinyl-4-aminoprolinyl-4-amino-{2-(acridine-9-carboxamide)acetyl}prolinyl-4-aminoprolinyl-4-amino-[(2-phenyl-quinoline-4-carboxamide)acetyl[prolinamide (Ac-Qgr-r-Agr-r-Pgr-NH₂)

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the first fluorophore (2-phenyl-quinoline-4-carboxylic acid (3 eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and acetylated with Ac₂O (10 eq) and DIEA (10 eq). The resin was then treated with 20% piperidine in DMF and Boc-Amp(Fmoc)-OH (3 eq) was coupled using DIPCDI (3 eq) and HOBt (3 eq). Then the Boc group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the second fluorophore (acridine-9-carboxylic acid, 3 eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and acetylated with Ac₂O (10 eq) and DIEA (10 eq). The resin was then treated with 20% piperidine in DMF and Boc-Amp(Fmoc)-OH (3 eq) was coupled using DIPCDI (3 eq) and HOBt (3 eq). Then the Boc group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the third fluorophore (10H-Indolo[3,2-b]quinoline-11-carboxylic acid, (3 eq)) was coupled to the resin using TBTU (3 eq) and DIEA (6 eq). Finally, the resin was treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. HPLC solutions were solvent A: H₂O containing 0.045% of TFA. Solvent B: acetonitrile containing 0.036% of TFA. Column: reverse-phase Symmetry C₁₈ (150×4.6 mm) 5 m, with UV detection at 220 nm. Flow rate 1 ml/min linear gradient from 0 to 100 in B. Retention time and purity in the HPLC: 5.2 min, 82%. MS (electrospray): found 1557.64 [M+H⁺], expected for C₈₄H₈₄N₁₈O₁₄ 1555.65.

### Example 65

### N⁴-Acetyl-4-amino-{2-(acridine-9-carboxamide)acetyl}prolinyl-4-aminoprolinyl-4-amino-[(2-phenyl-quinoline-4-carboxamide)acetyl]prolinamide-4-aminoprolinyl-4-amino-{2-(10H-indolo[3,2-b]quinoline-11-carboxamide)acetyl}prolinamide (Ac-Agr-r-Pgr-r-Qgr-NH₂)

This compound was synthesized following the same protocol described for example 64, but replacing the first fluorophore by 10H-indolo[3,2-b]quinoline-11-carboxylic acid (3 eq), the second one by 2-phenyl-quinoline-4-carboxylic acid (3 eq) and the third one by acridine-9-carboxylic acid (3 eq). The desired product was characterized by UV-spectra and mass spectrometry. Retention time and purity in the HPLC (see conditions in example 83): 5.2 min, 96%. MS (electrospray): found 1557.14 [M+H⁺], expected for C₈₃H₈₂N₁₈O₁₄ 1555.65.

### Example 66

### N⁴-Acetyl-4-amino-{2-(10H-indolo[3,2-b]quinoline-11-carboxamide)acetyl}prolinyl-4-aminoprolinyl-4-aminoprolinyl-4-amino-{2-(acridine-9-carboxamide)acetyl}prolinyl-4-aminoprolinyl-4-aminoprolinyl-4-amino-[(2-phenyl-quinoline-4-carboxamide)acetyl]prolinamide (Ac-Qgr-r-r-Agr-r-r-Pgr-NH₂)

Boc-Amp(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the first fluorophore (2-phenyl-quinoline-4-carboxylic acid (3 eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. The resin was treated again with 20% piperidine in DMF and Boc-Amp(Fmoc)-OH (3 eq) was coupled using DIPCDI (3 eq) and HOBt (3 eq) as coupling reagents. Then Boc protecting groups were removed with 40% TFA in DCM, the resin was treated with 5% DIEA in DCM and acetylated with Ac₂O (10 eq) and DIEA (10 eq). The resin was then treated with 20% piperidine in DMF and Boc-Amp(Fmoc)-OH (3 eq) was coupled using DIPCDI (3 eq) and HOBt (3 eq). Then the Boc group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the second fluorophore (acridine-9-carboxylic acid, 3eq)) was coupled to the resin using TBTU (3 eq) and DIEA (6 eq). The resin was then treated with 20% piperidine in DMF and Boc-Amp(Fmoc)-OH (3 eq) was coupled using DIPCDI (3 eq) and HOBt (3 eq) as coupling reagents. The resin was treated again with 20% piperidine in DMF and Boc-Amp(Fmoc)-OH (3 eq) was coupled using DIPCDI (3 eq) and HOBt (3 eq) as coupling reagents. Then Boc protecting groups were removed with 40% TFA in DCM, the resin was treated with 5% DIEA in DCM and acetylated with Ac₂O (10 eq) and DIEA (10 eq). The resin was then treated with 20% piperidine in DMF and Boc-Amp(Fmoc)-OH (3 eq) was coupled using DIPCDI (3 eq) and HOBt (3 eq). Then the Boc group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the third fluorophore (10H-indolo[3,2-b]quinoline-11-carboxylic acid, (3 eq)) was coupled to the resin using TBTU (3 eq) and DIEA (6 eq). Finally, the resin was treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 83): 5.1 min, 96%. MS (electrospray): found 1865.85 [M+H⁺], expected for C₉₇H₁₀₂N₂₂O₁₈ 1863.98.

### Example 67

### N⁴-Acetyl-4-amino-{2-(acridine-9-carboxamide)acetyl}prolinyl-4-aminoprolinyl-4-aminoprolinyl-4-amino-[(2-phenyl-quinoline-4-carboxamide)acetyl[prolinamide-4-aminoprolinyl-4-aminoprolinyl-4-amino-{2-(10H-indolo[3,2-b]quinoline-11-carboxamide)acetyl}prolinamide (Ac-Agr-r-r-Pgr-r-r-Qgr-NH₂)

This compound was synthesized following the same protocol described for example 66, but replacing the first fluorophore by 10H-indolo[3,2-b]quinoline-11-carboxylic acid (3 eq), the second one by 2-phenyl-quinoline-4-carboxylic acid (3 eq) and the third one by acridine-9-carboxylic acid (3 eq). The desired product was characterized by UV-spectra and mass spectrometry. Retention time and purity in the HPLC (see conditions in example 83): 5.2 min, 99%. MS (electrospray): found 1865.17 [M+H⁺], expected for C₉₇H₁₀₂N₂₂O₁₈ 1863.98.

### Example 68

### N⁴-Acetyl-5-N-{2-(acridine-9-carboxamide)acetyl}ornithinyl-5-N-{2-(acridine-9-carboxamide)acetyl}ornithinyl-5-N-{2-(acridine-9-carboxamide)acetyl}ornithinamide (Ac-Ago-Ago-Ago-NH₂)

Boc-Orn(Fmoc)-OH (3 eq) was coupled to the MBHA resin using DIPCDI (3 eq) and HOBt (3 eq) as coupling reagents. The resin was then treated with 20% piperidine in DMF and the second molecule of Boc-Orn(Fmoc)-OH (3 eq) was coupled using the same coupling reagents. The resin was treated again with 20% piperidine in DMF and the third Boc-Orn(Fmoc)-OH (3 eq) was coupled using the same coupling reagents. Then Boc protecting groups were removed with 40% TFA in DCM, the resin was treated with 5% DIEA in DCM and the spacer (Boc-Gly-OH, 15 eq) was introduced at this α-amino position using DIPCDI (15 eq) and HOBt (15 eq) as coupling reagents. After that, Boc group was removed and acridine-9-carboxylic acid (9 eq) was coupled to the resin using TBTU (9 eq) and DIEA (18 eq) for the coupling. The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 83): 4.3 min, 91%. MS (electrospray): found 1189.00 [M+H⁺], expected for C₆₅H₆₅N₁₃O₁₀ 1188.29.

### Example 69

### N⁴-Acetyl-5-N-{2-(acridine-9-carboxamide)acetyl}ornithinyl-5-N-{2-(10H-indolo[3,2-b]quinoline-11-carboxamide)acetyl}ornithinyl-5-N-{2-(acridine-9-carboxamide)acetyl}ornithinamide (Ac-Ago-Qgo-Ago-NH₂)

Boc-Orn(Fmoc)-OH (3 eq) was coupled to the MBHA resin using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the first fluorophore (acridine-9-carboxylic acid (3 eq)) was coupled to the resin using TBTU (3 eq) and DIEA (6 eq). The resin was then treated with 20% piperidine in DMF and Boc-Orn(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the second fluorophore (10H-indolo[3,2-b]quinoline-11-carboxylic acid (3 eq)) was coupled to the resin using TBTU (3 eq) and DIEA (6 eq). The resin was then treated with 20% piperidine in DMF and Boc-Orn(Fmoc)-OH (3 eq) was coupled using DIPCDI (3 eq) and HOBt (3 eq) as coupling reagents. Then Boc protecting group was removed with 40% TFA in DCM, the resin was treated with 5% DIEA in DCM and the spacer (Boc-Gly-OH, 5 eq) was introduced at this α-amino position using DIPCDI (5 eq) and HOBt (5 eq) as coupling reagents. After that, Boc group was removed and the third fluorophore (acridine-9-carboxylic acid (3 eq)) was coupled to the resin using TBTU (3 eq) and DIEA (6 eq). The resin was then treated with 20% piperidine in DMF and the N-terminal was acetylated using Ac₂O-DIEA (5 eq:5 eq). The final compound was cleaved with anhydrous HF at 0° C. during 1 h using anisole as scavenger. Then the HF was evaporated and the oligomer precipitated with cold diethyl ether, filtered and extracted with AcOH:H₂O (90:10) and the solvent was evaporated. The crude was dissolved in H₂O/ACN and lyophilised. The desired product was characterized by UV-spectra and mass spectrometry. Retention time and purity in the HPLC (see conditions in example 83): 4.6 min, 95%. MS (electrospray): found 1227.90 [M+H⁺], expected for C₆₇H₆₆N₁₄O₁₀ 1227.33.

### Example 70

### N⁴-Acetyl-5-N-{2-(acridine-9-carboxamide)acetyl}ornithinyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinamide (Ac-Ago-Qgo-Qgo-NH₂)

This compound was synthesized following the same protocol described for example 69, but replacing the first fluorophore by 10H-indolo[3,2-b]quinoline-11-carboxylic acid (3 eq). The desired product was characterized by UV-spectra and mass spectrometry. Retention time and purity in the HPLC (see conditions in example 83): 4.8 min, 92%. MS (electrospray): found 643.30 [(M+2H)/2²⁺], expected for C₆₉H₆₇N₁₅O₁₀ 1227.33.

### Example 71

### N⁴-Acetyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinamide Ac-Qgo-Qgo-Qgo-NH₂

This compound was synthesized following the same protocol described for example 68, but replacing the fluorophore by 10H-indolo[3,2-b]quinoline-11-carboxylic acid (3 eq). The desired product was characterized by UV-spectra and mass spectrometry. Retention time and purity in the HPLC (see conditions in example 83): 5.0 min, 84%. MS (electrospray): found 1305.90 [M+H⁺], expected for C₇₁H₆₈N₁₆O₁₀ 1305.40.

Fluorescent Properties of the New Compounds.

The excitation and emission wavelengths of the new compounds are described in Table 1. Optimal excitation wavelengths range from 247 to 433 nm and the corresponding emission wavelengths range from 392 to 505 nm. The quindoline derivatives with aminoproline backbone have the higher emission wavelengths. The phenylquinoline derivatives have the lower emission wavelengths.

TABLE 1
UV and Fluorescence properties of the new compounds.

| | | Fluorescence | Fluorescence |
|---|---|---|---|
| | | Excitation | emission |
| Compound | UV (λ, max) | (nm) | (nm) |
| 2a, Qut, Ex. 3 | 276, 306, 347, 400 | 346 | 402 |
| 2b, Act. Ex, 4 | 249, 343, 359, 385 | 358 | 439 |
| 2c, Qgt. Ex. 5 | 275, 303, 347, 401 | 374 | 446 |
| 2d, Agt, Ex. 6 | 250, 342, 359, 385 | 247 | 440 |
| 2e, Pht, Ex. 13 | 260, 328 | 326 | 377 |
| 2f, Nct, Ex. 16 | 272, 331, 351 | 261 | 456 |
| 5a, Aca, Ex. 18 | 264, 392, 410, 433 | 433 | 448, 470 |
| 5b, Cra, Ex. 21 | 281, 329, 409, 426 | 340 | 469 |
| Ex 27, Act-p-Qut | 252, 277, 349 | 358 | 438 |
| Ex 28, Qut-p-Qut | 272, 346, 396 | 346 | 495 |
| Ex 29, Act-p-Qut-p- | 251, 274, 349 | 353 | 440 |
| Qut | | | |
| Ex 30, Act-(p-Act)₅ | 247, 363 | 350 | 438 |
| Ex 31, Act-p-Cra | 252, 283, 342, 429 | 353 | 441 |
| Ex 32, Act-ps-Cra | 252, 283, 342, 395, | 359 | 448 |
| | 429 | | |
| Ex 33, Aca-p-Aca | 265, 412, 434 | 359 | 440 |
| Ex 34, Pht-p-Act | 252, 330 | 252 | 437 |
| Ex 35, Pht-p-Pht | 260, 330 | 330 | 395 |
| Ex 36, Act-p-Pht | 252, 339, 362 | 252 | 403 |
| Ex 37, Pht-p-Qut | 265, 337, 404 | 342 | 492 |
| Ex 38, Act-p-Qut-p- | 248, 280, 360 | 359 | 438 |
| Agt | | | |
| Ex 41, Qut-p-Cra | 276, 343, 407 | 275 | 476 |
| Ex 42, Qut-p-Qut-p- | 273, 349, 399 | 359 | 499 |
| Qut | | | |
| Ex 43, Act-p-Act-p- | 246, 361 | 360 | 440 |
| Act | | | |
| Ex 44, Act-p-Qut-p- | 252, 281, 360 | 359 | 438 |
| Act | | | |
| Ex 45, Act-p-Qut-p- | 253, 272, 335 | 271 | 449 |
| Nct | | | |
| Ex 46, Agp-Agp | 252, 360 | 248 | 441 |
| Ex 47, Agp-Agp-Agp | 252, 360 | 360 | 436 |
| Ex 48, Qgp-Qgp | 275, 350 | 349 | 495 |
| Ex 50, Agr | 252, 360 | 282 | 439 |
| Ex 51, Qgr | 224, 276, 348, 401 | 347 | 502 |
| Ex 52, Agr-Agr | 251, 361 | 274 | 450 |
| Ex 53, Qgr-Qgr | 223, 276, 349 | 348 | 396, 504 |
| Ex 54, Agr-Qgn | 223, 252, 276, 349 | 273 | 498 |
| Ex 55, Qgr-Agr | 252, 277, 350 | 308 | 500 |
| Ex 56, Agr-Agr-Agr | 245, 361 | 275 | 440 |
| Ex 57, Qgr-Qgr-Qgr | 224, 275, 349 | 348 | 505 |
| Ex 58, Agr-Qgr-Agr | 252, 279, 353 | 275 | 503 |
| Ex 60, Qgr-Agr-Agr | 250, 278, 360 | 309 | 456 |
| Ex 61, Qgr-Agr-Qgr | 223, 253, 277, 350 | 348 | 488 |
| Ex 63, Agr-Qgr-Qgr | 253, 275, 352 | 272 | 503 |
| Ex 64, Qgr-Agr-Pgr | 253, 346 | 347 | 488 |
| Ex 65, Agr-r-Pgr-r- | 252, 342 | 249 | 442 |
| Qgr | | | |
| Ex 66, Qgr-r-r-Agr-r- | 253, 338 | 269 | 432 |
| r-Pgr | | | |
| Ex 67, Agr-r-r-Pgr-r- | 252, 301, 341, 360 | 265 | 451 |
| r-Qgr | | | |
| Ex 68, Ago-Ago-Ago | 252, 361 | 360 | 435 |
| Ex 69, Ago-Qgo-Ago | 252, 280, 359 | 357 | 441 |
| Ex 70, Ago-Qgo-Qgo | 225, 252, 275, 351 | 248 | 446 |
| Ex 71, Qgo-Qgo- | 275, 351 | 338 | 466 |
| Qgo | | | |

DNA-Duplex Stabilization Properties

Solid supports carrying oligomers with L-threoninol and 3-aminopropan-1,2-diol backbones were introduced on a DNA synthesizer and oligonucleotides were assembled using standard protocols. After the assembly of the desired DNA sequence, supports were treated with concentrated ammonia at 55° C. for a minimum of 6 hrs yielding oligonucleotides carrying oligomers of fluorophores at the 3′-end.

Two oligonucleotide sequences were synthetized (sequence 1: 5′-TTCCGGAA-3′ sequence 2: 5′-CCAATTGG-3′) carrying different fluorophores at the 3′-end. Solid supports containing dimer and trimer fluorophores (prepared in examples 28-31) and standard protocols for oligonucleotide synthesis were used in order to obtain these modified oligonucleotides (Table 2). Ammonia deprotection was performed at 55° C. for 6 hours. The resulting solutions were concentrated to dryness. Then, the residue was dissolved in water and desalted through a NAP-10 column. Fractions containing oligonucleotides were analysed by HPLC and characterized by UV and MS spectrometry.

TABLE 2
TTCCGGAA-p-Qut, found: 2819.2; expected for
C₉₈H₁₁₇N₃₃O₅₁P₈ 2820.6
TTCCGGAA-p-Agt (1d), found: 2837.1; expected for
C₉₈H₁₁₉N₃₃O₅₂P₈ 2838.6
CCAATTGG-p-Act (1b), found: 2779.9; expected for
C₉₆H₁₁₆N₃₂O₅₁P₈ 2781.6
TTCCGGAA-p-Cra, found: 2789.7; expected for
C₉₇H₁₁₅N₃₃O₅₀P₈ 2790.6
CCAATTGG-p-Cra, found: 2790.4; expected for
C₉₇H₁₁₅N₃₃O₅₀P₈ 2790.6
CCAATTGG-p-Qut, found: 2817.8; expected for
C₉₈H₁₁₇N₃₃O₅₁P₈ 2820.6
CCAATTGG-Agt (1d), found 2836.1; expected for
C₉₈H₁₁₉N₃₃O₅₂P₈ 2838.6
CCAATTGG-p-Act-p-Qut-p-Qut, found 3601.4;
C₁₃₆H₁₅₂N₃₈O₆₁P₁₀ 3604.3
TTCCGGAA-p-Qut-p-Qut, found 3228.0;
C₁₁₈H₁₃₆N₃₆O₅₆P₉ 3232.9
TTCCGGAA-p-Pht, found 2807.0;
C₉₈H₁₁₈N₃₂O₅₁P₈ 2807.6
TTCCGGAA-p-Act-p-Pht, found 3178.2;
C₁₁₆H₁₃₅N₃₄O₅₆P₉ 3179.9
TTCCGGAA-p-Nct, found 2834.9;
C₁₀₀H₁₁₈N₃₃O₅₁P₈ 2845.7
TTCCGGAA-p-Qgt, found 2778.0;
C₁₀₀H₁₂₀N₃₄O₅₂P₈ 2877.7
TTCCGGAA-p-Act-p-Qut-p-Nct, found 3616.4;
C₁₃₈H₁₃₄N₃₈O₆₁P₁₀ 3610.14
TTCCGGAA-p-Act-p-Qut, found 3191.3 (M+ Na⁺),
C₁₁₆H₁₁₄N₃₅O₅₆P₉ 3172.8

Next, the fluorescent properties of duplexes carrying fluorophores at the 3′-end was determined and the results are shown in Table 3:

TABLE 3

| | Excitation | Emission |
|---|---|---|
| Sequence (5′-3′) | (nm) | (nm) |
| TTCCGGAA-p-Qut (1a) | 349 | 492 |
| TTCCGGAA-p-Act-p-Qut-p-Qut | 352 | 436 |
| TTCCGGAA-p-Act-p-Qut | 351 | 485 |
| TTCCGGAA-p-Qut-p-Qut | 349 | 490 |
| TTCCGGAA-p-Act-p-Pht | 336 | 440 |
| TTCCGGAA-p-Nct | 267 | 442 |
| TTCCGGAA-p-Qgt | 350 | 491 |
| CCAATTGG-p- Agt (1d) | 350 | 438 |
| CCAATTGG- p-Qut (1a) | 273 | 493 |
| CCAATTGG -p-Act-p-Qut-p-Qut | 248 | 440 |
| CCAATTGG- p-Qut-p-Qut | 359 | 496 |

As it is derived from the obtained data, when linked to oligonucleotides the fluorescent properties are preserved.

Melting Experiments

Appropriate oligonucleotides were dissolved in a solution containing 1 M NaCl, 10 mM sodium phosphate buffer of pH=7. UV Absorption spectra and melting experiments (absorbance vs temperature) were recorded in 1-cm path length cell by using a spectrophotometer, with a temperature controller and a programmed temperature increase rate of 1° C./min. Melting curves were recorded at 260 nm, and melting temperatures were measured at the maximum of the first derivatives of the melting curves. Results are shown in Table 4.

TABLE 4

| Sequence (5′-3′) | Tm(° C.) | ΔTm (° C.) |
|---|---|---|
| TTCCGGAA | 35 | — |
| TTCCGGAA-p-Act (1b) | 47 | +12 |
| TTCCGGAA-p-Agt (1d) | 47 | +12 |
| TTCCGGAA-p-Qut (1a) | 44 | +9 |
| TTCCGGAA-p-Act-p-Qut-p-Qut | 44 | +9 |
| TTCCGGAA-p-Act-p-Qut | 44 | +9 |
| TTCCGGAA-p-Qut-p-Qut | 48 | +13 |
| TTCCGGAA-p-Pht | 41 | +6 |
| TTCCGGAA-p-Act-p-Pht | 43 | +8 |
| TTCCGGAA-p-Nct | 46 | +11 |
| TTCCGGAA-p-Qgt | 50 | +15 |
| TTCCGGAA-p-Act-p-Qut-p-Nct | 60 | +25 |
| CCAATTGG | 37 | — |
| CCAATTGG-p-Agt (1d) | 40 | +3 |
| CCAATTGG -p-Act-p-Qut-p-Qut | 39 | +2 |
| CCAATTGG- p-Qut-p-Qut | 39 | +2 |

As it is derived from the different assays, the presence of the polymer on the 3′-end of the oligonucleotide increases the melting temperature up to 25 degrees of the duplex indicating an interaction of the fluorophore with the duplex structure of DNA. Said increase in the melting temperature indicates an increase in the stabilization of the oligonucleotide to which the polymer of the invention is attached.

Competitive Dialysis

Compounds that bind nucleic acids may show preference for certain sequences. This preference can be used for the detection of specific nucleic acid sequences. One of the methods used to analyse the sequence specificity of DNA-binding compounds is the competition dialysis (c.f. Ren, J. et al., “Sequence and structural selectivity of nucleic acid binding ligands”, Biochemistry, 1999, vol. 38, pp 1607-1675). In this method different nucleic acid structures are dialyzed against a common compound solution. After equilibration the nucleic acid sequence with a higher affinity is able to retain a higher amount of the compound. The amount of compound present in each DNA sequence is measured by fluorescence.

We analysed the sequence specificity of the following polymers of the invention: Aca-p-Aca, QgpQgp(npa), Act-(p-Act)₅, Act-p-Qut-p-Agt, Act-p-Qut-p-Act, Act-p-Qut-p-Nct, Qut-p-Qut-p-Qut, Act-p-Qut-p-Qut, and Act-p-Act-p-Act for DNA sequences:
duplex with an alternating CG sequence (5′-CGCGCG-T₄-CGCGCG-3′);
duplex with a contiguous CG sequence (5′- CCCGGG-T₄-CCCGGG-3′);
duplex with an alternating AT sequence (5′-ATATATAT-T₄-ATATATAT);
duplex with a contiguous AT sequence (5′-AAAATTTT-T₄-AAAATTTT);
a parallel triplex (5′-T₂₀-3′-3′-T₂₀-5′+A₂₀); and
a G-quadruplex (thrombin-binding aptamer: 5′-GGTTGGTGTGGTTGG-3′ (SEQ ID NO 1); (c.f. Bock, L. C. et al., “Selection of single-stranded DNA molecules that bind and inhibit human thrombin”, Nature, 1992, vol. 355, pp 564-566).

Said DNA sequences have been used in order to determine: 1) the affinity of each one of the polymers for a specific tandem of nucleotides (CG, AT, GT); and 2) if the affinity for a specific tandem of nucleotides is affected by its distribution into the sequence (i.e., if the tandem is alternated or contiguous in the sequence).

Furthermore, the inventors analyzed the binding properties of two dimers of the invention, Act-p-Act and Act-p-Qut, in front of several DNA sequences which are known in the state in the art to be biologically-relevant G-quadruplex structures:
5′- GGTTGGTGTGGTTGG-3′, (SEQ ID NO: 1 supra) as thrombin-binding aptamer: G-quadruplex;
5′- GGGGAGGGTGGGGAGG GTGGGGAAGGTGGGG-3′ (SEQ ID NO: 2) as a fragment of the promoter region of c-myc oncogen (c.f. Siddiqui-Jain A. et al., “Direct evidence for a G-quadruplex in a promoter region and its targeting with a small molecule to repress c-myc transcription”, Proc. Natl. Acad. Sci. USA, 2002, v. 99, p. 11593-11598);
5′-TAGGGTTAGGGTTAGGG TTAGGGT-3′ (SEQ ID NO: 3) as a repeat of human telomeric DNA, (c.f. Parkinson G. N. et al. “Crystal structure of parallel quadruplex from human telomeric DNA”, Nature, 2002, v. 417, p. 876-880);
5′-CGGGCGCGGGAGGAAGGGGG CGGG-3′ (SEQ ID NO: 4) as a the bcl2 promoter region fragment (c.f. Dai J. et al., “NMR solution structure of the major G-quadruplex structure formed in the human bcl2 promoter region”, Nucleic Acids Res., 2006, v. 34, p. 5133-5144);
5′TGGGGGT-3′ as a the tetramolecular parallel quadruplex (c.f. Gros

J. et al., “Guanine are a quartet's best friend: impact of base substitutions on the kinetics and stability of tetramolecular quadruplex”, Nucleic Acids Res., 2007, v.35, p. 3064-3075); and
5′-CCCGCCCCCTTCCTCCCGCGC CCG -3′ (SEQ ID NO: 5) as a bcl2 promoter region fragment that at neutral pH is mostly single-stranded but form an i-motif structure in acidic conditions (c.f. Khan et al., “Solution equilibria of the i-motif-forming region upstream of the B-cell lymphoma 2P1 promoter”, Biochemie, 2007, v. 89, p. 1562-1572).

All the DNA sequences used for the competition assays has been prepared following standard solid-phase protocols using phosphoramidite derivatives (c.f M. H. Caruthers et al., “Chemical synthesis of deoxyoligonucleotides by the phosphoramidite method” Methods Enzymol., 1987, vol. 154, pp 287-313).

The competition dialysis assay was performed placing into a beaker 200 ml of dialysate solution containing 1 μM of ligand in 2 mM sodium phosphate, 2 mM EDTA and 0.185 M NaCl pH 7.0. A volume of 0.1 ml (at 1 mg/ml) of each DNA samples was pipeted into a separate 0.1 ml dialysis unit (Mini Slide-A-lyzer 3.5K, Pierce). The dialysis units were placed in the beaker containing the dialysate solution using a floating device. The contents were allowed to equilibrate with continuous stirring for 24 hours at room temperature. At the end of the equilibration period, DNA samples were carefully removed to eppendorf tubes and were taken to a final concentration of 1% (w/v) sodium dodecyl sulfate (SDS). The concentration of polymer within each dialysis unit was then determined by fluorescence spectroscopy. The free polymer concentration was determined using an aliquot of the dialysate solution as well as with the solution coming from a blank (without DNA) dialysis unit.

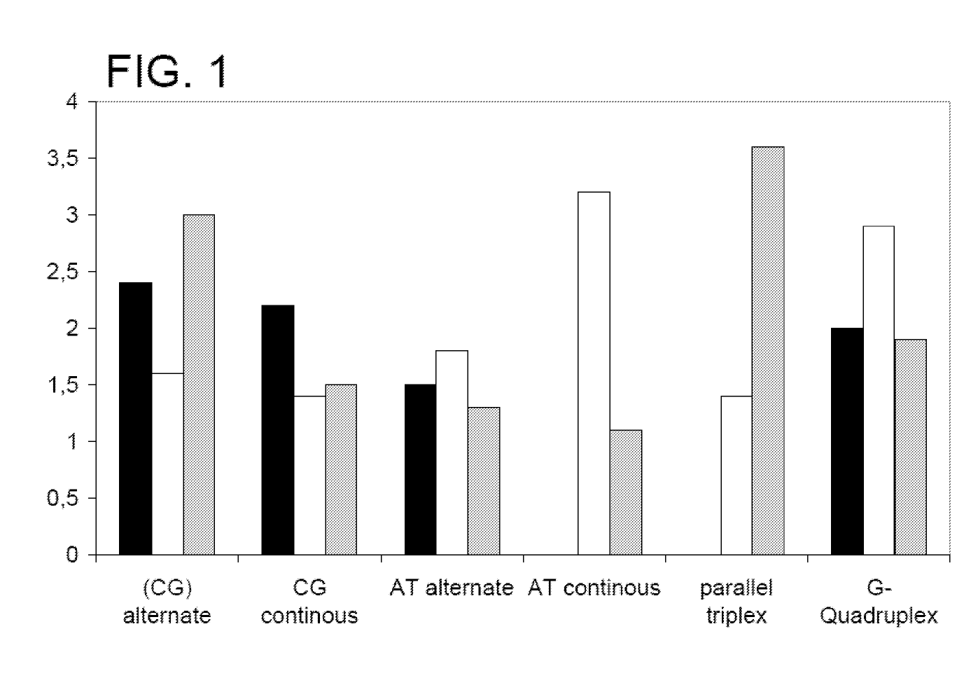

FIG. 1-3 summarize the results obtained. The concentration of the polymer (which is determined by fluorescence spectroscopy) indicates its specificity for a specific sequence: the more higher is the concentration, more specific is for said sequence.

In FIG. 1 it is remarkable the high affinity of the dimmer Qgp-Qgp to contiguous AT sequence and G-quadruplex and the high affinity of hexamer to the parallel triplex sequence.

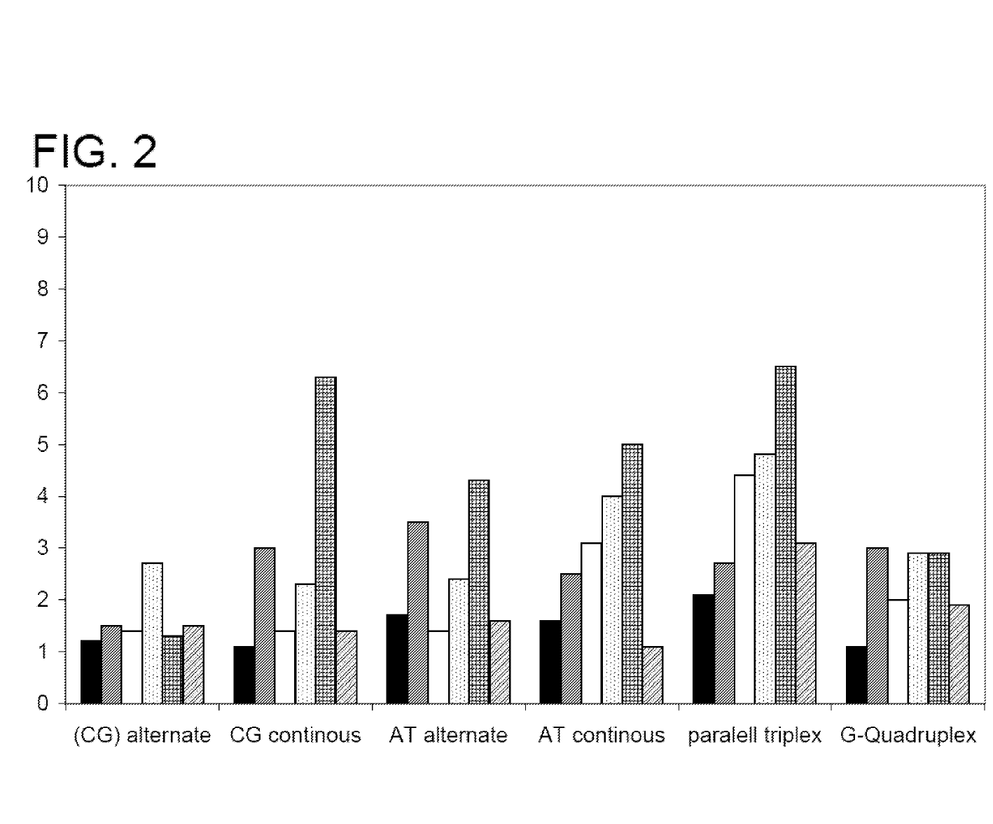

FIG. 2 it is shown that the amount of fluorescence found, when trimers were used in the dialysis experiments, is higher than that obtained using dimers and hexamer. This is indicative that a trimer or quadruplex may be optimal for the DNA binding. It is worth to mention the higher affinity of Act-p-Qut-p-Act, Act-p-Qut-p-Qut and Qut-p-Qut-p-Qut for AT rich sequences, contiguous CG sequence and parallel triplex because these type of nucleic acid sequences are not usual binding sites for known intercalating drugs.

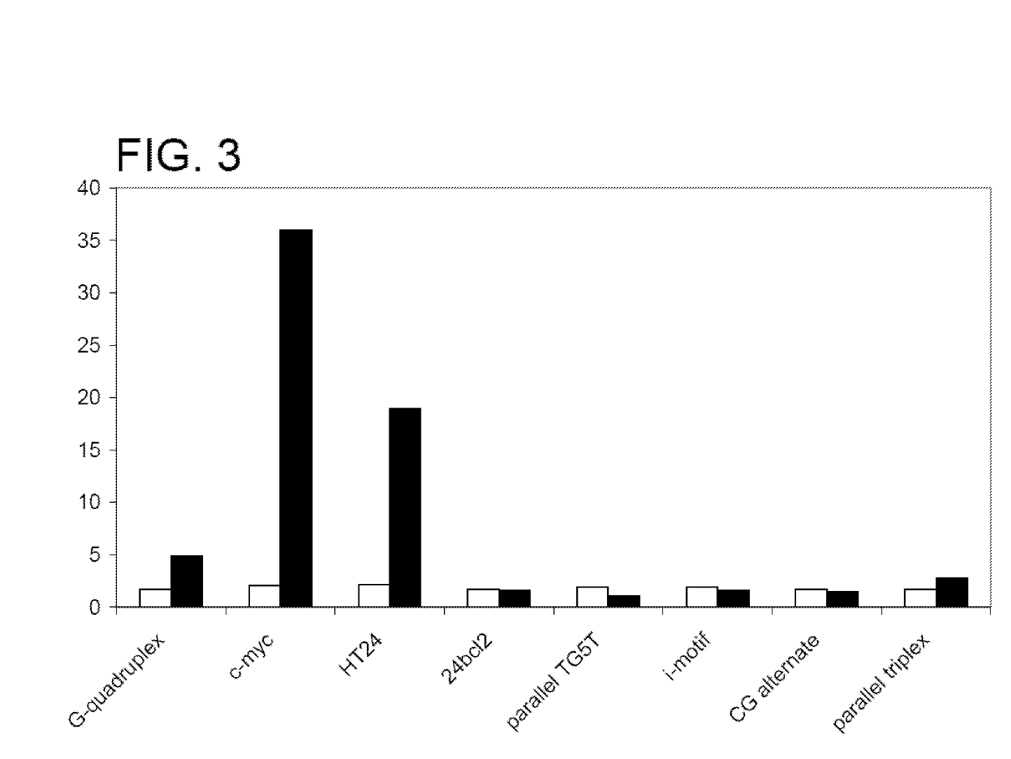

FIG. 3 shows the dramatic differences between the binding affinities of these dimers. Act-p-Qut has a very high affinity to c-myc promoter region and human telomere G-quadruplex structures.

In summary, the binding properties of these molecules show distinct affinities for model nucleic acid sequences. Preliminary results show that the presence of the negatively charged phosphodiester or amide backbone does not compromise the binding to DNA. On the contrary a high affinity is observed for multistranded nucleic acid sequences (triplex and quadruplex). The higher affinity for quadruplex structures is of special interest as to use the compounds described in this invention as fluorescent probes for the detection of biologically-relevant quadruplex structures.

Moreover the derivatives described in this invention can also be used to introduce fluorescent compounds into synthetic DNA, RNA and peptides using solid-phase protocols.

1. A polymer composed by two to ten monomers of formula (I) 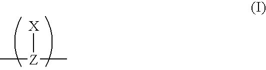
wherein:
X is a radical of formula (II) 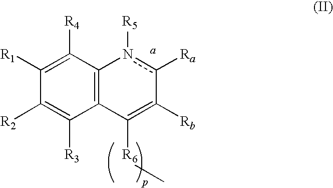
wherein
—R₅ is an electron pair or a (C₁-C₃)-alkyl radical;
—Rₐ and —R_{b} are radicals independently selected from the group consisting of H, (C₁-C₄)-alkyl, (C₁-C₄)-alkoxy, (C₁-C₄)-alkylamino, phenyl, F, Cl, Br, amino, hydroxy, and nitro or
—Rₐ and —R_{b} are fused forming with the carbon atoms to which they are attached a ring of formula (III) 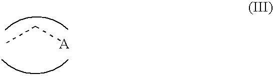
with the condition that
(i) when —R₅ is an electron pair, a is a N═C double bond, and Rₐ and R_{b} are fused forming the ring 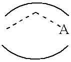
said ring being a biradical selected from (IIIa) and (IIIb) 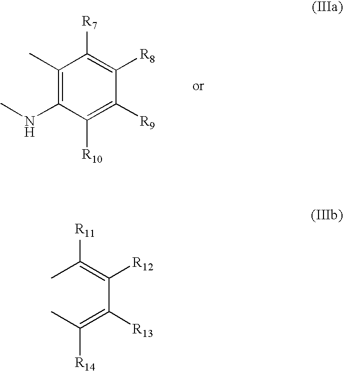
thus, radical (II) is (IIa) or (IIb) respectively 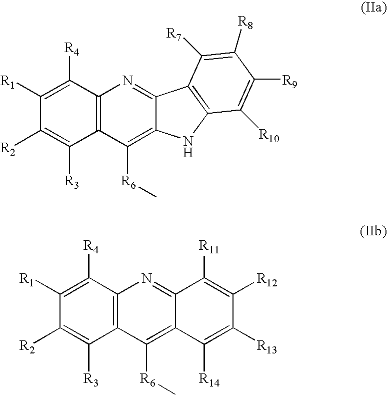
(ii) when —R₅ is a (C₁-C₃)-alkyl radical, a is a N—C single bond and Rₐ and R_{b} are fused forming the ring 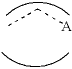
said ring being a biradical 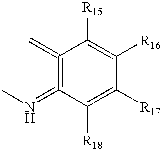
thus, the radical (II) is (IIc); 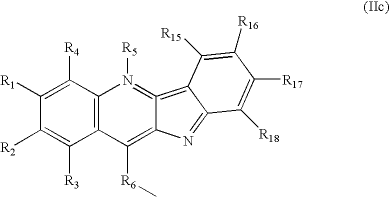
R₁-R₄ and R₇-R₁₈ represent radicals, same or different, selected from the group consisting of H, (C₁-C₄)-alkyl, (C₁-C₄)-alkylamino, phenyl, F, Cl, Br, amino, hydroxy, and nitro;
p is an integer from 0 to 1;
R₆ is a biradical selected from the group consisting of —CO—; —CONH(CH₂)ₘCO—; and —CO[NHCHR″CO]ₘ—, wherein —R″ are side chains radicals, same or different, corresponding to natural aminoacids; and m is an integer from 1 to 3;
Z is a triradical of formula (IV) 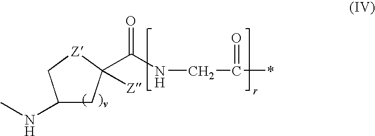
wherein
r is an integer from 0 to 1;
v is an integer from 0 to 1;
Z′ is a triradical selected from —CH₂— and nitrogen;
Z″ is H,
with the proviso that:
(a) when Z′ is nitrogen, forming an amide bound with R₆, then Z″ is hydrogen and v is an integer from 0 to 1, and
(b) when Z″ is —NH—, forming an amide group with R₆, then Z′ is —CH₂— and v=0,
or of formula (V): 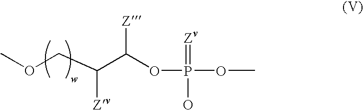
wherein
Z″' is selected from —CH₃ and —CH₂NH—,
Z^{iv} is selected from H and NH,
Z^{v} is selected from S and O atom,
W is an integer from 0 to 1,
with the proviso that
(c) when R₆ is bound to Z′″, then Z′″ is —CH₂NH—, Z^{iv} is hydrogen and w is 0; and
(d) when Z^{iv} is —NH— forming an amide bound with R₆, Z′″ is —CH₃ and w is 1; and
and wherein the monomers of formula (I) are linked through the triradical Z, forming an amide or phosphate bound.

2. The polymer according to claim 1 wherein the triradical Z group corresponds to the formula (IV).

3. The polymer according to claim 1 wherein the triradical Z group corresponds to the formula (V).

4. The polymer according to claim 1, wherein the triradical Z of formula (IV) is selected from the group consisting of: 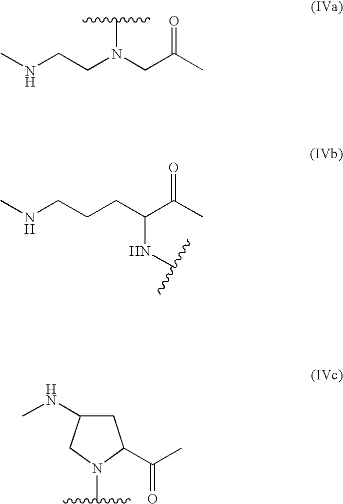
wherein the symbol [symbol] indicates the position through which triradical Z is attached to the radical X.

5. The polymer according to claim 1, wherein the triradical Z of formula (V) is selected from the group consisting of: 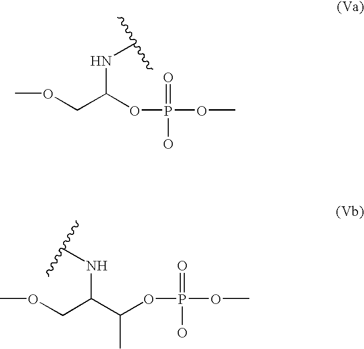
wherein the symbol [symbol] indicates the position through which radical Z is attached to radical X.

6. The polymer according to claim 1, wherein the radical X of formula (II) is selected from the group consisting of: 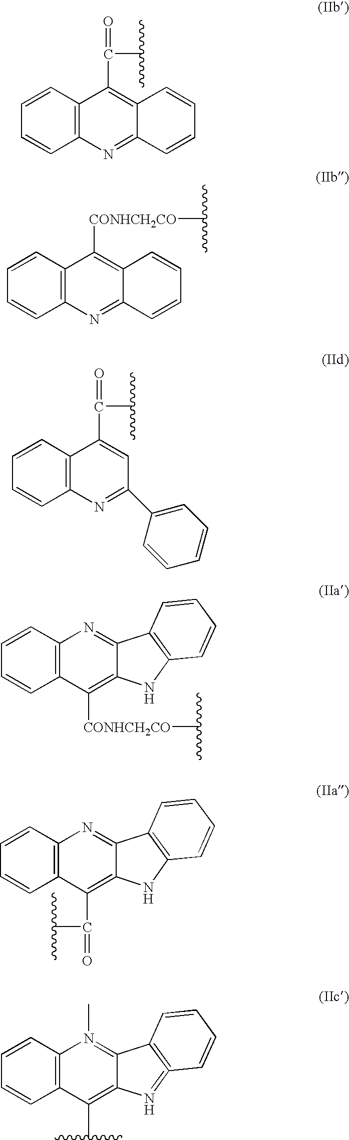
wherein the symbol [symbol] indicates the position through which radical X is attached by its radical R₆ to the triradical Z.

7. The polymer as defined in claim 1 wherein R₆ is —CO—.

8. The polymer as defined in claim 1 wherein R₆ is —CONH—.

9. The polymer as defined in claim 1 wherein R₆ is —CO[NHCHR″CO]ₘ—, being —R″ the side chain radical corresponding to the aminoacid selected from glicine and proline; and m is an integer from 1 to 3.

10. The polymer according to claim 1, which is a homopolimer.

11. The polymer according to claim 1, which is a heteropolymer.

12. The polymer according to claim 1, which is selected from the group consisting of:
O-{2-N-(Acridine-9-carbamoyl)-(1-hydroxybut-3-yl)} and O-{2-N-(10H-indolo[3,2-d]quinoline-11-carbamoyl)-3-hydroxybut-1-yl} phosphate;
O-{2-N-(10H-indolo[3,2-d]quinoline-11-carbamoyl)-(1-hydroxybut-3-yl)} and O-(2-N-{10H-indolo[3,2-d]quinoline-11-carbamoyl }-3-hydroxybut-1-yl) phosphate;
O-{2-N-(Acridine-9-carbamoyl)-(1-hydroxybut-3-yl)} phosphate (3-1) O-{2-N-(10H-indolo[3,2- d]quinoline-11-carbamoyl)-3-oxybut-1-yl} phosphate (3-1) O-(2-N-{10H-indolo[3,2-d]quinoline-11-carbamoyl)}-3-hydroxybut-1-yl);
O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-3-hydroxybut-1-yl};
O-{2-N-(Acridine-9-carbamoyl)-(1-hydroxybut-3-yl)} and O-{3-(5-methyl-5H-indolo[3,2b]quinolin-11-ylamino-(S)-(2-hydroxyprop-3 -yl} phosphate;
O-{2-N-(Acridine-9-carbamoyl)-(1-hydroxybut-3-yl)} and O-{3-(5-methyl-5H-indolo[3,2b]quinolin-11-ylamino-(S)-(2-hydroxyprop-3-yl} phosphorotioate;
O-[(R)-3-(Acridin-9-ylamino)-1-hydroxy-propane-2-yl]-phosphate (2-1) O-{2-N-(acridine-9-ylamino)-2-oxyprop-1-yl} phosphate(2-4) 4-hydroxybutiramide;
O-{2-N-(2-phenylquinoline-4-carbamoyl-1-hydroxybutan-3-yl)} and O-{2-N-(acridine-9-carbamoyl)-3-hydroxybut-1-yl} phosphate;
O-{2-N-(2-phenylquinoline-4-carbamoyl-1-hydroxybutan-3-yl)} and O-{2-N-(2-phenylquinoline-4-carbamoyl-3-hydroxybutan-1-yl)} phosphate;
O-{2-N-(acridine-9-carbamoyl)-1-hydroxybut-3-yl} and O-{2-N-(2-phenylquinoline-4-carbamoyl-3-hydroxybutan-1-yl) } phosphate;
O-{2-N-(2-phenylquinoline-4-carbamoyl-1-hydroxybutan-3-yl)} and O-(2-N-{10H-indolo[3,2-d]quinoline-11-carbamoyl}-3-hydroxybut-1-yl);
O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carboxamido)acetamido]-(3-hydroxybut-1-yl)};
(2S, 3R)-2-((9H-Fluoren-9-yl)methyloxycarbonyl)amino)butane-1-(4,4′-dimethoxytrityloxy)butan-3-ol;
(2S, 3R)-2-((9H-Fluoren-9-yl)methyloxycarbonyl)amino)butane-1-(4,4′-dimethoxytrityloxy)butan-3-yl N,N-diisopropylamino-2-cyanoethyl phosphoramidite;
O-{2-N-(10H-indolo[3,2-d]quinoline-11-carbamoyl)-3-oxybut-1-yl} phosphate (3-1) O-{3-(5-methyl-5H-indolo[3,2-b]quinolin-11-ylamino-(S)-(2hydroxylprop-3-yl);
O-{2-N-(10H-indolo[3,2-d]quinoline-11-carbamoyl)-3-oxybut-1-yl} phosphate (3-1) O-(2-N-(10H-indolo[3,2-d]quinoline-11-carbamoyl)-3-oxybut-1-yl} phosphate (3-1) O-(2-N-{10H-indolo[3,2-d]quinoline-11-carbamoyl}-3-hydroxybut-1-yl);
O-{2-N-(Acridine-9-carbamoyl)-(1-hydroxybut-3-yl)} phosphate (3-1) O-{2-N-(acridine-9-carbamoyl)-(1-hydroxybut-3-yl)} phosphate (3-1) O-{2-N-(acridine-9-carbamoyl)-(3-hydroxybut-1-yl)};
O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl} phosphate (3-1) O-{2-N-(acridine-9-carbamoyl)-(3-hydroxybut-1-yl)};
O-{2-N-(Acridine-9-carbamoyl)-(1-hydroxybut-3-yl)} phosphate (3-1) O-{2-N-(10H-indolo[3,2-d]quinoline-11-carbamoyl)-3-oxybut-1-yl } phosphate (3-1) O-{2-N-(5H-indolo[2,3-b]quinolin-5-yl)acetamido-3-hydroxybut-1-yl);
Acetyl-{2-[Acridine-9-carbonyl)-amino]acetyl}-(2-aminoethyl)-glycyl-{2-[Acridine-9-carbonyl)-amino]acetyl}-(2-aminoethyl)-N-6-hydroxyhexyl glycynamide;
Acetyl-{2-[Acridine-9-carbonyl)-amino]acetyl}-(2-aminoethyl)-glycyl-{2-[Acridine-9-carbonyl)-amino]-acetyl}-(2-aminoethyl)-glycyl {2-[Acridine-9-carbonyl)-amino]-acetyl}-(2-aminoethyl)-N-6-hydroxyhexyl glycynamide;
Acetyl-{2-N-(10H-indolo[3,2-d]quinoline-11-carbonyl)-amino]acetyl}-(2-aminoethyl)-glycyl-{2-N-(10H-indolo[3,2-d]quinoline-11-carbonyl)-amino]acetyl}-(2-aminoethyl)-N-6-hydroxyhexyl glycynamide;
N-[2-(Acridine-9-carboxamide)]-4-N-[2-(acridine-9-carboxamide)prolinamide;
N-[2-(Acridine-9-carboxamide)acetyl]-4-N-acetamidoprolinamide;
N-{2-(10H-indolo[3,2-b]quinoline-11-carboxamide)acetyl}-4-N-acetamidoprolinamide;
N⁴-Acetyl-4-amino-{2-(acridine-9-carboxamide)acetyl}prolinyl-4-amino-{2-(acridine-9-carboxamide)acetyl}prolinamide;
N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinamide;
10H-Indolo[3,2-b]quinoline-11-carboxylic acid {2-[4-acetylamino-2-(1-{2-[(acridine-9-carbonyl)-amino]-acetyl -5-carbamoyl-pyrrolidin-3 -ylcarbamoyl)-pyrrolidin-1-y]-2-oxo-ethyl}-amide;
10H-Indolo[3,2-b]quinoline-11-carboxylic acid (2-{4-[(4-acetylamino-1-{2-[(acridine-9-carbonyl)-amino]-acetyl}-pyrrolidine-2-carbonyl)-amino]-2-carbamoyl-pyrrolidin-1-yl}-2-oxo-ethyl)-amide;
N⁴-Acetyl-4-amino-{2-(acridine-9-carboxamide)acetyl}prolinyl-4-amino-{2-(acridine-9-carboxamide)acetyl}prolinyl-4-amino-2-(acridine-9-carboxamide)acetyl}prolinamide;
N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-{{2-(10H-indolo[3,2-b]quinoline-11-carboxamide)acetyl}prolinamide;
N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(acridine-9-carboxamide)acetyl}prolinamide;
N⁴-Acetyl-4-amino-{2-(10H-indolo[3,2-b]quinoline-11-carboxamide)acetyl}prolinyl-4-amino-{2-(10H-indolo[3,2-b]quinoline-11-carboxamide)acetyl}prolinyl-4-amino-{2-(acridine-9-carboxamide)acetyl}prolinamide;
N⁴-Acetyl-4-amino-{2-(10H-indolo[3,2-b]quinoline-11-carboxamide)acetyl}prolinyl-4-amino-{2-(acridine-9-carboxamide)acetyl}prolinyl-4-amino-{2-(acridine-9-carboxamide)acetyl}prolinamide;
N⁴-Acetyl-4-amino-{2(10H-indolo[3,2-b]quinoline-11-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide;
N⁴-Acetyl-4-amino-{2-(acridine-9-carboxamide)acetyl}prolinyl-4-amino-{2-(acridine-9-carboxamide)acetyl}prolinyl-4-amino-{2-(10H-indolo[3,2-b]quinoline-11-carboxamide)acetyl}prolinamide;
N⁴-Acetyl-4-amino-{2-(acridine-9-carboxamide)acetyl}prolinyl-4-amino-{2-(10H-indolo[3,2-b]quinoline-11-carboxamide)acetyl}proliny-4-amino-{2-(10H-indolo[3,2-b]quinoline-11-carboxamide)acetyl}prolinamide;
N⁴-Acetyl-4-amino-{2-(10H-indolo[3,2-b]quinoline-11-carboxamide)acetyl}prolinyl-4-aminoprolinyl-4-amino-{2-(acridine-9-carboxamide)acetyl}prolinyl-4-aminoprolinyl-4-amino-[(2-phenyl-quinoline-4-carboxamide)acetyl]prolinamide;
N⁴-Acetyl-4-amino-{2-(acridine-9-carboxamide)acetyl}prolinyl-4-aminoprolinyl-4-amino-[(2-phenyl-quinoline-4-carboxamide)acetyl]prolinamide-4-aminoprolinyl-4-amino-{2-(10H-indolo[3,2-b]quinoline-11-carboxamide)acetyl}prolinamide;
N⁴-Acetyl-4-amino-{2-(10H-indolo[3,2-b]quinoline-11-carboxamide)acetyl}prolinyl-4-aminoprolinyl-4-aminoprolinyl-4-amino-{2-(acridine-9-carboxamide)acetyl}prolinyl-4-aminoprolinyl-4-aminoprolinyl-4-amino-[(2-phenyl-quinoline-4-carboxamide)acetyl]prolinamide;
N⁴-Acetyl-4-amino-{2-(acridine-9-carboxamide)acetyl}prolinyl-4-aminoprolinyl-4-aminoprolinyl-4-amino-[(2-phenyl-quinoline-4-carboxamide)acetyl]prolinamide-4-aminoprolinyl-4-aminoprolinyl-4-amino-{2-(10H-indolo[3,2-b]quinoline-11-carboxamide)acetyl}prolinamide;
N⁴-Acetyl-5-N-{2-(acridine-9-carboxamide)acetyl}ornithinyl-5-N-{2-(acridine-9-carboxamide)acetyl}ornithinyl-5-N-{2-(acridine-9-carboxamide)acetyl}ornithinamide;
N⁴-Acetyl-5-N-{2-(acridine-9-carboxamide)acetyl}ornithinyl-5-N-{2-(10H-indolo[3,2-b]quinoline-11-carboxamide)acetyl}ornithinyl-5-N-{2-(acridine-9-carboxamide)acetyl}ornithinamide;
N⁴-Acetyl-5-N-{2-(acridine-9-carboxamide)acetyl}ornithinyl-5-N-{2(10H-indolo[3,2-b]quinoline-11-carboxamide)acetyl}ornithinyl-5-N-{2-(10H-indolo[3,2-b]quinoline-11-carboxamide)acetyl}ornithinamide; and
N⁴-Acetyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinyl-5-N-{2-(10H-indolo[3,2-b]quinoline-11-carboxamide)acetyl}ornithinamide.

13. A process for preparing a polymer as defined in claim 1, characterized in that it is carried out coupling compounds of formula (VI): 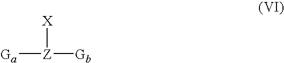
wherein
X and Z are as defined in any one of the preceding claims, and
Gₐ and G_{b} are protecting groups,
on a solid-phase support.

14. The process as defined in claim 13 wherein Gₐ and G_{b} are selected from dimethoxytrityl, —P(OCH₂CH₂CN)—N(isopropyl)₂ and t-butoxy carbonyl protecting groups.

15. A compound of formula (VI): 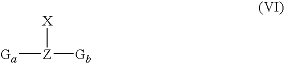
X is a radical of formula (II) 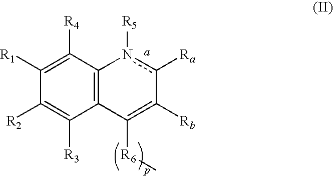
wherein
—R₅ is an electron pair or a (C₁-C₃)-alkyl radical;
—Rₐ and —R_{b} are radicals independently selected from the group consisting of H, (C₁-C₄)-alkyl, (C₁-C₄)-alkoxy, (C₁-C₄)-alkylamino, phenyl, F, Cl, Br, amino, hydroxy, and nitro or
—Rₐ and —R_{b} are fused forming with the carbon atoms to which they are attached a ring of formula (III) 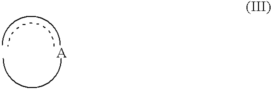
with the condition that
(i) when —R₅ is an electron pair, a is a N═C double bond, and Rₐ and R_{b} are fused forming the ring 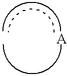
said ring being a biradical selected from (IIIa) and (IIIb) 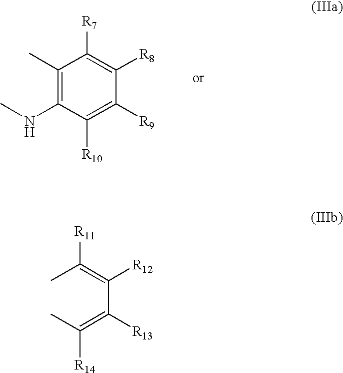
thus, radical (II) is (IIa) or (IIb) respectively 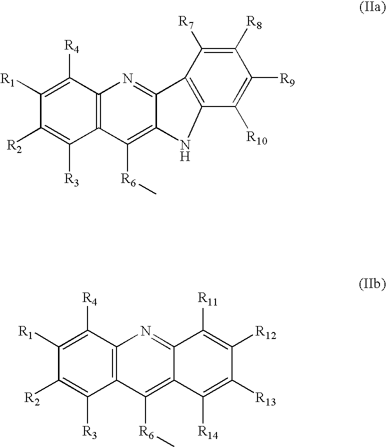
(ii) when —R₅ is a (C₁-C₃)-alkyl radical, a is a N—C single bond and Rₐ and R_{b} are fused forming the ring 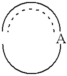
said ring being a biradical 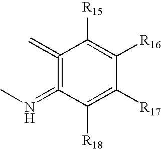
thus, the radical (II) is (IIc); 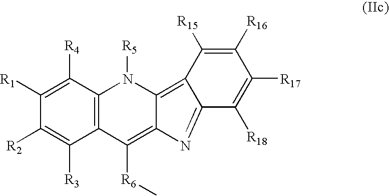
R₁-R₄ and R₇-R₁₈ represent radicals, same or different, selected from the group consisting of H, (C₁-C₄)-alkyl, (C₁-C₄)-alkylamino, phenyl, F, Cl, Br, amino, hydroxy, and nitro;
p is an integer from 0 to 1;
R₆ is a biradical selected from the group consisting of —CO—; —CONH(CH₂)ₘCO—; and —CO[NHCHR″CO]ₘ—, wherein —R″ are side chains radicals, same or different, corresponding to natural aminoacids; and m is an integer from 1 to 3;
Z is a triradical of formula (IV) 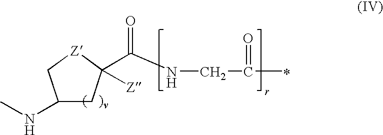
wherein
r is an integer from 0 to 1;
v is an integer from 0 to 1;
Z′ is a triradical selected from —CH₂— and nitrogen;
Z″ is H
with the proviso that:
(a) when Z′ is nitrogen, forming an amide bound with R₆, then Z″ is hydrogen and v is an integer from 0 to 1, and
(b) when Z″ is —NH—, forming an amide group with R₆, then Z′ is —CH₂— and v=0,
or of formula (V): 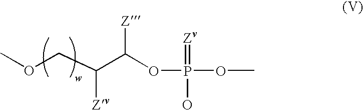
wherein
Z′″ is selected from —CH₃ and —CH₂NH—,
Z^{iv} is selected from H and NH,
Z^{v} is selected from S and O atom,
W is an integer from 0 to 1,
with the proviso that
(c) when R₆ is bound to Z′″, then Z′″ is —CH₂NH—, Z^{iv} is hydrogen and w is 0; and
(d) when Z^{iv} is —NH— forming an amide bound with R₆, Z′″ is —CH₃ and w is 1;
and wherein the monomers of formula (I) are linked through the triradical Z, forming an amide or phosphate bound, and Gₐ and G_{b} are protecting groups.

16. A compound according to claim 15 which is linked to a solid support through one of the protecting groups Gₐ and G_{b}.

17. (canceled)

18. A method of preparing a fluorescently labeled nucleic acid molecule which method comprises:
incorporating at least one polymer according to claim 1 into a RNA or DNA molecule under conditions sufficient to incorporate said polymer.

19. A method of detecting a target nucleic acid in a sample to be tested which method comprises:
contacting the target nucleic acid with a nucleic acid probe comprising at least one polymer according to claim 1 for a time under conditions sufficient to permit hybridization between said target and said probe; and detecting said hybridization.

20. A DNA analysis procedure, wherein the improvement comprises:
utilizing the polymer as defined in claim 1 in the DNA analysis procedure.

21. A fluorescence resonance energy transfer procedure, wherein the improvement comprises:
utilizing the polymer as defined in claim 1 in the fluorescence resonance energy transfer procedure.
